(19) United States
(12) Patent Application Publication
(10) Pub. No.: US 2025/0101420 A1
(43) Pub. Date: Mar. 27, 2025

(54) MODIFIED siRNA FOR SELECTIVELY INHIBITING EXPRESSION OF MUTANT FUS

(71) Applicant: OHARA PHARMACEUTICAL CO., LTD., Shiga (JP)

(72) Inventors: Naoki Morita, Shiga (JP); Kaede Hashiseko, Shiga (JP); Sumire Okada, Shiga (JP); Yoshinobu Yamamoto, Shiga (JP)

(21) Appl. No.: 18/730,190

(22) PCT Filed: Mar. 6, 2023
(86) PCT No.: PCT/JP2023/008197

§ 371 Date: Jul. 18, 2024

(30) Foreign Application Priority Data
Mar. 8, 2022 (JP) 2022-035727

(51) Int. Cl. C12N 15/113; A61P 25/28

(52) U.S. Cl. CPC C12N 15/113; A61P 25/28; C12N 2310/11; C12N 2310/14; C12N 2310/315; C12N 2310/321; C12N 2310/322

(57) ABSTRACT

[Problems]

To provide a chemically modified siRNA that has improved stability against RNase enzymes and selectively exhibits silencing of FUS P525L mutation which is a causative gene of ALS.

[Solution]

Provided is a chemically modified siRNA comprising a sense strand and an antisense strand, or a salt thereof, wherein: the antisense strand includes a complementary or substantially complementary region in a portion of a mRNA that codes FUS P525L mutation protein; the complementary region has a nucleotide length of 19 to 21; and the siRNA contains at least one substitution selected from the group consisting of a 2′-F-nucleotide, a 2′-OMe-nucleotide, a nucleotide in which a 2′-O atom and a 4′-O atom are bridged by methylene, a 2′-deoxynucleotide, and a phosphorothioate bond that forms an internucleotide bond.

### TECHNICAL FIELD

The present invention relates to chemically modified siRNA which has improved stability against RNases and selectively exhibits silencing of FUS (fused in sarcoma) P525L mutation, the causative gene of amyotrophic lateral sclerosis (ALS) and a pharmaceutical composition containing the chemically modified siRNA.

### BACKGROUND TECHNIQUE

Amyotrophic lateral sclerosis (ALS), also known as Lou Gehrig's disease, is the most fatal progressive neurodegenerative disease characterized by the predominant loss of motor neurons (MN) in the primary motor cortex, brainstem, and spinal cord. The loss of motor neurons disrupts basal movements such as breathing, and typically leads to the death of the patient within two to five years after diagnosis. The progressive deterioration of the patient's motor function severely reduces their ability to breathe, making some form of respiratory support necessary for their survival. Other symptoms may include decrease in muscle strength of hands, arms, legs, or swallowing muscles. In addition, some patients may also develop frontotemporal dementia (FTD). The most common age for ALS is between 50 and 70 years old, making it a common disease among the elderly.

ALS can be broadly classified into two types: sporadic ALS and familial ALS, most cases thereof being sporadic ALS, which is non-hereditary. Familial ALS is a disease with a relatively small number of patients, accounting for approximately 5-10% of all ALS cases.

The pathogenesis of ALS onset is complex. It is generally believed to be a complex genetic disease caused by mutations in multiple genes coupled with environmental exposures. More than a dozen causative genes have been identified as factors related to the onset of ALS, including SOD1 (Cu²⁺/Zn²⁺ superoxide dismutase), TDP-43 (TAR DNA-binding protein-43 kD), FUS (fused in sarcoma), ANG (angiogenin), ATXN2 (ataxin-2), VCP (valosin-containing protein), OPTN (optineurin), and C9orf72 (chromosome 9 open reading frame 72). However, the exact mechanism of motor neuron degeneration remains unclear.

FUS is known to be the second most frequent causative gene for familial ALS after SOD1. The FUS, the causative gene for ALS6 linked to chromosome 16, is an RNA-binding protein identified in 2009, and is known to be a causative gene that is more prevalent in relatively young people in familial ALS (non-patent document 1).

FUS shuttles between the nucleus and the cytoplasm, performing important RNA metabolic functions such as DNA repair and splicing regulation. It is known that mutations in FUS cause abnormal aggregation in the cytoplasm, and the following two hypotheses have been proposed as factors behind the onset of familial ALS. The first hypothesis is loss of function, in which RNA metabolism, which should take place in the nucleus, cannot be performed normally. The second hypothesis is toxic gain of function due to aggregation of mutant protein into cytoplasm.

The C-terminus of the FUS protein contains a nuclear localization signal, and if a mutation occurs in this region, the affinity with transportin, a nuclear transport receptor, may decrease. In such cases, it is considered that nuclear localization of FUS can no longer performed normally, and mutated FUS accumulates in the cytoplasm. An investigation into the location of FUS mutations revealed that there were many mutations in sites of the nuclear localization signal, such as P495X, G507D, K510R/E, S513P, R514G/S, R514S, G515C, H517Q/P, R518G/K, R521G/C/H, R522G, R524W/T/S, and P525L. Furthermore, it is known that among the mutations in the sites of nuclear localization signal, P525L mutation is common in juvenile ALS that develops in people in their teens and twenties, and most of these patients die within two years of onset. Currently, no therapeutic agent has been developed.

Wild-type FUS has an important RNA metabolism function as described above, and it is reported that the FUS knockout in forebrain cortical neurons of mice results in a decrease in interaction with SFPQ, a splicing factor (splicing factor proline and glutamine rich) of RNA and changes to tau isoform. Therefore, high selectivity to mutant FUS is essential in the development of therapeutic agents for the ALS caused by FUS mutations.

On the other hand, reports on siRNA targeting genes with point mutations include, for example, siRNA targeting EGFR (epithelial growth factor receptor) G356D mutation (patent document 1), siRNA targeting APP (amyloid precursor protein) V337M mutation (non-patent document 2), and siRNA targeting SOD1 G85R mutation (non-patent document 3). However, there is no teaching and suggestion regarding the chemically modified siRNA that selectively exhibits silencing of FUS P525L mutation while improving the stability against RNases in plasma that cleave RNA strands nonspecifically. Although a method has been proposed for diagnosing ALS or a genetic predisposition to ALS using specific genetic markers and treating or preventing ALS using siRNA molecules that reduce the expression of mutant FUS (patent document 2), there is no description on specific siRNA sequence.

### PRIOR ART DOCUMENTS

### Patent Documents

1. Pamphlet of WO2011/158924
2. Pamphlet of WO2010/011283

### Non-Patent Documents

1. Kwiatkowski T J, et al. Mutations in the FUS/TLS gene on chromosome 16 cause familial amyotrophic lateral sclerosis. Science, 2009; 323: 1205-1208.
2. Miller V M, et al. Targeting Alzheimer's disease genes with RNA interference: an efficient strategy for silencing mutant alleles. Nucleic Acids Res, 2004; 32: 661-668.
3. Ding H, et al. Selective silencing by RNAi of a dominant allele that causes amyotrophic lateral sclerosis. Aging Cell, 2003; 2: 209-217.

### SUMMARY OF THE INVENTION

### Problems to be Solved by the Invention

An object of the present invention is to provide chemically modified siRNA that has improved the stability against RNase in plasma that nonspecifically cleave RNA strands and selectively exhibits silencing of FUS P525L mutation, a causative gene for ALS. A further object of the present invention is to provide a pharmaceutical composition containing the chemically modified siRNA.

### Means for Solving the Problems

To solve the problem that siRNA that selectively exhibits silencing of FUS P525L mutation, the causative gene of ALS, has low stability against RNase in plasma, the inventors replaced the nucleotides constituting the siRNA with 2′-F-nucleotides and 2′-OMe-nucleotides. The 2′-F-nucleotides and 2′-OMe-nucleotides were arranged alternately in the RNA strand of the siRNA. By arranging two or four consecutive 2′-F-nucleotides or 2′-OMe-nucleotides, the inventors found an siRNA that maintains high RNAi activity equivalent to that of naked siRNA and high selectivity for the mRNA encoding the FUS P525L mutation while dramatically improving the stability against RNases at the site of cleavage of the RNA strand by Ago2, one of the proteins constituting the RNA-induced silencing complex (RISC) present in cells, thereby completing the present invention.

The present disclosure includes the following features.
[1]A chemically modified siRNA or a salt thereof consisting of a sense strand and an antisense strand, wherein the antisense strand comprises a region complementary or substantially complementary to a part of mRNA encoding FUS P525L mutation protein, the complementary region is 19 to 21 nucleotides in length, the siRNA comprises at least one substitution selected from the group consisting of 2′-F-nucleotides, 2′-OMe-nucleotides, nucleotides in which a 2-O atom and a 4-C atom are bridged with methylene, 2′-deoxy-nucleotides, and phosphorothioate bonds that form internucleotide linkage.
[2] The chemically modified siRNA or a salt thereof described in [1], comprising a motif of two or four consecutive 2′-F-nucleotides or 2′-OMe-nucleotides at or adjacent to the site of cleavage of the RNA strand by Ago2, and comprising 2′-F-nucleotides and 2′-OMe-nucleotides alternately along the RNA strand except the motif.
[3] The chemically modified siRNA or a salt thereof described in [2], wherein the siRNA is represented by formula (I) below:

(I)
Sense strand:
5′-(YX)a-(YY)b-(XY)c-(XX)d-(YX)e-(YY)f-(XY)g-
(XYX)h-(YXY)i-3′
Antisense strand:
3′-(YX)j-(XY)k-(XY)a-(XX)b-(YX)c-(YY)d-(XY)e-
(XX)f-(YX)g-(Y)h-(X)i-5′

(Wherein, X and Y are 2′-F-nucleotides and 2′-OMe-nucleotides, respectively;
a, b, c, d, e, f, g, h, i, j, and k are each independently an integer of 0 to 4;
(a, b, c, j, k) are (0, 0, 4, 1, 0), (0, 1, 3, 0, 1), (0, 2, 2, 0, 1), (1, 1, 2, 0, 1), (2, 1, 1, 0, 1), (1, 2, 1, 0, 1), (2, 2, 0, 0, 1), or (3, 1, 0, 0, 1) in this order;
(e, f, g, h, i) are (4, 0, 0, 0, 1), (3, 1, 0, 1, 0), (2, 2, 0, 1, 0), (2, 1, 1, 1, 0), (1, 1, 2, 1, 0), (1, 2, 1, 1, 0), (0, 2, 2, 1, 0), or (0, 1, 3, 1, 0) in this order when d is 1;
(e, f, g, h, i) are (3, 0, 0, 0, 1), (2, 1, 0, 1, 0), (1, 2, 0, 1, 0), (1, 1, 1, 1, 0), (0, 2, 1, 1, 0), or (0, 1, 2, 1, 0) in this order when d is 2);
and is 21 nucleotides in length.
[4] The chemically modified siRNA or a salt thereof described in [2] or [3], selected from the group consisting of the double-stranded RNA consisting of the sense strand of SEQ ID NO: 5 and the antisense strand of SEQ ID NO: 6, the double-stranded RNA consisting of the sense strand of SEQ ID NO: 13 and the antisense strand of SEQ ID NO: 14, the double-stranded RNA consisting of the sense strand of SEQ ID NO: 15 and the antisense strand of SEQ ID NO: 16, the double-stranded RNA consisting of the sense strand of SEQ ID NO: 17 and the antisense strand of SEQ ID NO: 18, the double-stranded RNA consisting of the sense strand of SEQ ID NO: 19 and the antisense strand of SEQ ID NO: 20, the double-stranded RNA consisting of the sense strand of SEQ ID NO: 21 and the antisense strand of SEQ ID NO: 22, the double-stranded RNA consisting of the sense strand of SEQ ID NO: 23 and the antisense strand of SEQ ID NO: 24, the double-stranded RNA consisting of the sense strand of SEQ ID NO: 35 and the antisense strand of SEQ ID NO: 36, the double-stranded RNA consisting of the sense strand of SEQ ID NO: 37 and the antisense strand of SEQ ID NO: 38, the double-stranded RNA consisting of the sense strand of SEQ ID NO: 39 and the antisense strand of SEQ ID NO: 40, the double-stranded RNA consisting of the sense strand of SEQ ID NO: 41 and the antisense strand of SEQ ID NO: 42, the double-stranded RNA consisting of the sense strand of SEQ ID NO: 43 and the antisense strand of SEQ ID NO: 44, the double-stranded RNA consisting of the sense strand of SEQ ID NO: 45 and the antisense strand of SEQ ID NO: 46, the double-stranded RNA consisting of the sense strand of SEQ ID NO: 47 and the antisense strand of SEQ ID NO: 48, the double-stranded RNA consisting of the sense strand of SEQ ID NO: 49 and the antisense strand of SEQ ID NO: 50, the double-stranded RNA consisting of the sense strand of SEQ ID NO: 51 and the antisense strand of SEQ ID NO: 52, the double-stranded RNA consisting of the sense strand of SEQ ID NO: 53 and the antisense strand of SEQ ID NO: 54, the double-stranded RNA consisting of the sense strand of SEQ ID NO: 55 and the antisense strand of SEQ ID NO: 56, the double-stranded RNA consisting of the sense strand of SEQ ID NO: 57 and the antisense strand of SEQ ID NO: 58, the double-stranded RNA consisting of the sense strand of SEQ ID NO: 59 and the antisense strand of SEQ ID NO: 60, the double-stranded RNA consisting of the sense strand of SEQ ID NO: 63 and the antisense strand of SEQ ID NO: 64, the double-stranded RNA consisting of the sense strand of SEQ ID NO: 67 and the antisense strand of SEQ ID NO: 68, the double-stranded RNA consisting of the sense strand of SEQ ID NO: 71 and the antisense strand of SEQ ID NO: 72, the double-stranded RNA consisting of the sense strand of SEQ ID NO: 73 and the antisense strand of SEQ ID NO: 74, the double-stranded RNA consisting of the sense strand of SEQ ID NO: 75 and the antisense strand of SEQ ID NO: 76, the double-stranded RNA consisting of the sense strand of SEQ ID NO: 79 and the antisense strand of SEQ ID NO: 80, the double-stranded RNA consisting of the sense strand of SEQ ID NO: 81 and the antisense strand of SEQ ID NO: 82, the double-stranded RNA consisting of the sense strand of SEQ ID NO: 85 and the antisense strand of SEQ ID NO: 86, the double-stranded RNA consisting of the sense strand of SEQ ID NO: 87 and the antisense strand of SEQ ID NO: 88, the double-stranded RNA consisting of the sense strand of SEQ ID NO: 89 and the antisense strand of SEQ ID NO: 90, the double-stranded RNA consisting of the sense strand of SEQ ID NO: 91 and the antisense strand of SEQ ID NO: 92, the double-stranded RNA consisting of the sense strand of SEQ ID NO: 93 and the antisense strand of SEQ ID NO: 94, the double-stranded RNA consisting of the sense strand of SEQ ID NO: 97 and the antisense strand of SEQ ID NO: 98, the double-stranded RNA consisting of the sense strand of SEQ ID NO: 99 and the antisense strand of SEQ ID NO: 100, the double-stranded RNA consisting of the sense strand of SEQ ID NO: 103 and the antisense strand of SEQ ID NO: 104, the double-stranded RNA consisting of the sense strand of SEQ ID NO: 105 and the antisense strand of SEQ ID NO: 106, the double-stranded RNA consisting of the sense strand of SEQ ID NO: 107 and the antisense strand of SEQ ID NO: 108, the double-stranded RNA consisting of the sense strand of SEQ ID NO: 109 and the antisense strand of SEQ ID NO: 110, the double-stranded RNA consisting of the sense strand of SEQ ID NO: 111 and the antisense strand of SEQ ID NO: 112, the double-stranded RNA consisting of the sense strand of SEQ ID NO: 113 and the antisense strand of SEQ ID NO: 114, the double-stranded RNA consisting of the sense strand of SEQ ID NO: 117 and the antisense strand of SEQ ID NO: 118, the double-stranded RNA consisting of the sense strand of SEQ ID NO: 119 and the antisense strand of SEQ ID NO: 120, the double-stranded RNA consisting of the sense strand of SEQ ID NO: 123 and the antisense strand of SEQ ID NO: 124, the double-stranded RNA consisting of the sense strand of SEQ ID NO: 125 and the antisense strand of SEQ ID NO: 126, the double-stranded RNA consisting of the sense strand of SEQ ID NO: 127 and the antisense strand of SEQ ID NO: 128, and the double-stranded RNA consisting of the sense strand of SEQ ID NO: 129 and the antisense strand of SEQ ID NO: 130.
[5] The chemically modified siRNA or a salt thereof described in [2] or [3], selected from the group consisting of the double-stranded RNA consisting of the sense strand of SEQ ID NO: 13 and the antisense strand of SEQ ID NO: 14, the double-stranded RNA consisting of the sense strand of SEQ ID NO: 21 and the antisense strand of SEQ ID NO: 22, the double-stranded RNA consisting of the sense strand of SEQ ID NO: 39 and the antisense strand of SEQ ID NO: 40, the double-stranded RNA consisting of the sense strand of SEQ ID NO: 43 and the antisense strand of SEQ ID NO: 44, the double-stranded RNA consisting of the sense strand of SEQ ID NO: 49 and the antisense strand of SEQ ID NO: 50, the double-stranded RNA consisting of the sense strand of SEQ ID NO: 53 and the antisense strand of SEQ ID NO: 54, the double-stranded RNA consisting of the sense strand of SEQ ID NO: 55 and the antisense strand of SEQ ID NO: 56, the double-stranded RNA consisting of the sense strand of SEQ ID NO: 57 and the antisense strand of SEQ ID NO: 58, the double-stranded RNA consisting of the sense strand of SEQ ID NO: 59 and the antisense strand of SEQ ID NO: 60, the double-stranded RNA consisting of the sense strand of SEQ ID NO: 87 and the antisense strand of SEQ ID NO: 88, the double-stranded RNA consisting of the sense strand of SEQ ID NO: 89 and the antisense strand of SEQ ID NO: 90, the double-stranded RNA consisting of the sense strand of SEQ ID NO: 91 and the antisense strand of SEQ ID NO: 92, the double-stranded RNA consisting of the sense strand of SEQ ID NO: 93 and the antisense strand of SEQ ID NO: 94, the double-stranded RNA consisting of the sense strand of SEQ ID NO: 111 and the antisense strand of SEQ ID NO: 112, the double-stranded RNA consisting of the sense strand of SEQ ID NO: 113 and the antisense strand of SEQ ID NO: 114, the double-stranded RNA consisting of the sense strand of SEQ ID NO: 123 and the antisense strand of SEQ ID NO: 124, the double-stranded RNA consisting of the sense strand of SEQ ID NO: 125 and the antisense strand of SEQ ID NO: 126, the double-stranded RNA consisting of the sense strand of SEQ ID NO: 127 and the antisense strand of SEQ ID NO: 128, and the double-stranded RNA consisting of the sense strand of SEQ ID NO: 129 and the antisense strand of SEQ ID NO: 130.
[6] The chemically modified siRNA or a salt thereof described in [1], selected from the group consisting of the double-stranded RNA consisting of the sense strand of SEQ ID NO: 3 and the antisense strand of SEQ ID NO: 4, the double-stranded RNA consisting of the sense strand of SEQ ID NO: 7 and the antisense strand of SEQ ID NO: 8, the double-stranded RNA consisting of the sense strand of SEQ ID NO: 9 and the antisense strand of SEQ ID NO: 10, the double-stranded RNA consisting of the sense strand of SEQ ID NO: 11 and the antisense strand of SEQ ID NO: 12, the double-stranded RNA consisting of the sense strand of SEQ ID NO: 25 and the antisense strand of SEQ ID NO: 26, the double-stranded RNA consisting of the sense strand of SEQ ID NO: 27 and the antisense strand of SEQ ID NO: 28, the double-stranded RNA consisting of the sense strand of SEQ ID NO: 29 and the antisense strand of SEQ ID NO: 30, the double-stranded RNA consisting of the sense strand of SEQ ID NO: 31 and the antisense strand of SEQ ID NO: 32, the double-stranded RNA consisting of the sense strand of SEQ ID NO: 33 and the antisense strand of SEQ ID NO: 34, the double-stranded RNA consisting of the sense strand of SEQ ID NO: 65 and the antisense strand of SEQ ID NO: 66, the double-stranded RNA consisting of the sense strand of SEQ ID NO: 83 and the antisense strand of SEQ ID NO: 84, the double-stranded RNA consisting of the sense strand of SEQ ID NO: 121 and the antisense strand of SEQ ID NO: 122.
[7] The chemically modified siRNA or a salt thereof described in any one of [1] to [6], where in the three nucleotides located at 5′-end and 3′-end of sense strands and antisense strands, the adjacent internucleotide linkage are phosphorothioate bonds.
[8] The chemically modified siRNA or a salt thereof described in any one of [1] to [7] for inhibiting the expression of FUS P525L mutation.
[9] The chemically modified siRNA or a salt thereof described in any one of [1] to [7] for selectively silencing of FUS P525L mutation without substantially silencing of wild-type FUS.
[10] An inhibitor against the expression of FUS P525L mutation, containing the chemically modified siRNA or a salt thereof described in any one of [1] to [9].
[11]A pharmaceutical composition for prevention or treatment of ALS, containing the chemically modified siRNA or a salt thereof described in any one of [1] to [10] as an active ingredient.
[12] The pharmaceutical composition described in [11], wherein the ALS is ALS having FUS P525L mutation.

### Effects of the Invention

According to the present invention, it is possible to provide a chemically modified siRNA or a salt thereof that has dramatically improved the stability against RNase while maintaining high RNAi activity equivalent to that of naked siRNA and high selectivity for the mRNA encoding the FUS P525L mutation.

By using the chemically modified siRNA or a salt thereof of the present disclosure, it is possible to selectively silencing of the FUS P525L mutation without substantially silencing of wild-type FUS. In addition, a pharmaceutical composition containing the chemically modified siRNA or a salt thereof of the present disclosure enables effective treatment of ALS or ALS having FUS P525L mutation.

### BRIEF DESCRIPTION OF DRAWINGS

FIG. 1 shows the effect of the chemically modified siRNA of the present disclosure with respect to the mRNA expression rate of wild-type FUS and FUS P525L mutation.

FIG. 2 shows the effect of the chemically modified siRNA of the present disclosure with respect to the mRNA expression rate of wild-type FUS and FUS P525L mutation.

FIG. 3 shows the effect of the chemically modified siRNA of the present disclosure with respect to the mRNA expression rate of wild-type FUS and FUS P525L mutation.

FIG. 4 shows the effect of the chemically modified siRNA of the present disclosure with respect to the mRNA expression rate of wild-type FUS and FUS P525L mutation.

FIG. 5 shows the effect of the chemically modified siRNA of the present disclosure with respect to the mRNA expression rate of wild-type FUS and FUS P525L mutation.

FIG. 6 shows the stability of naked siRNA in human serum.

FIG. 7 shows the stability of the chemically modified siRNA of the present disclosure in human serum.

### EMBODIMENTS FOR CARRYING OUT THE INVENTION

The chemically modified siRNA (small interfering RNA) of the present disclosure is a double-stranded RNA consisting of an RNA complementary to the mRNA transcribed from FUS P525L mutation, the causative gene of ALS (antisense strand), and an RNA complementary to the antisense strand (sense strand). The chemically modified siRNA can degrade the mRNA of FUS P525L mutation by RNA interference (RNAi) and selectively exhibits silencing of FUS P525L mutation involved in ALS.

The chemically modified siRNA of the present disclosure includes a region complementary or substantially complementary to a part of the mRNA encoding FUS P525L mutation, and the complementary region is 19 to 21 nucleotides in length. In addition, in some embodiments, the sense strands and antisense strands in the chemically modified siRNA of the present disclosure are each 19 to 26 nucleotides in length. In some embodiments, the chemically modified siRNA of the present disclosure is 19 to 23 nucleotides in length.

As used herein, the term “complementary” means that the sense strand and antisense strand of siRNA, or the antisense strand of siRNA and a target mRNA, are bound by hydrogen bonds formed by the complementary base moieties of opposing nucleotides. As used herein, the term “substantially complementary” refers to a situation in which one or several opposing nucleotides bind by forming base pairs as a whole oligonucleotide, although they are not complementary nucleotides.

The chemically modified siRNA of the present disclosure comprises at least one substitution selected from the group consisting of 2′-F-nucleotides, 2′-OMe-nucleotides, nucleotides in which a 2-O atom and a 4′-C atom are bridged with methylene (LNA), 2′-deoxy-nucleotides, and phosphorothioate bonds that form internucleotide linkage.

In some embodiments, the chemically modified siRNA of the present disclosure comprises a motif of two or four consecutive 2′-F-nucleotides or 2′-OMe-nucleotides at or adjacent to the site of cleavage of the RNA strand by Ago2, and comprises 2′-F-nucleotides and 2′-OMe-nucleotides alternately along the RNA strand except the motif. Ago2 (Argonaute 2) is one of the proteins constituting the RNA-induced silencing complex (RISC). siRNA is incorporated into RISC. After sense strands are removed, antisense strands recognize target mRNA, and the mRNA is cleaved by Ago2.

The chemically modified siRNA can be represented by formula (I) below

(I)
Sense strand:
5′-(YX)a-(YY)b-(XY)c-(XX)d-(YX)e-(YY)f-(XY)g-
(XYX)h-(YXY)i-3′
Antisense strand:
3′-(YX)j-(XY)k-(XY)a-(XX)b-(YX)c-(YY)d-(XY)e-
(XX)f-(YX)g-(Y)h-(X)i-5′

The above formula (I) represents a chemically modified siRNA or a salt thereof in a length of 21 nucleotides, wherein, X and Y are 2′-F-nucleotides and 2′-OMe-nucleotides, respectively; a, b, c, d, e, f, g, h, i, j, and k are each independently an integer of 0 to 4; (a, b, c, j, k) are (0, 0, 4, 1, 0), (0, 1, 3, 0, 1), (0, 2, 2, 0, 1), (1, 1, 2, 0, 1), (2, 1, 1, 0, 1), (1, 2, 1, 0, 1), (2, 2, 0, 0, 1), or (3, 1, 0, 0, 1) in this order; (e, f, g, h, i) are (4, 0, 0, 0, 1), (3, 1, 0, 1, 0), (2, 2, 0, 1, 0), (2, 1, 1, 1, 0), (1, 1, 2, 1, 0), (1, 2, 1, 1, 0), (0, 2, 2, 1, 0), or (0, 1, 3, 1, 0) in this order when d is 1; (e, f, g, h, i) are (3, 0, 0, 0, 1), (2, 1, 0, 1, 0), (1, 2, 0, 1, 0), (1, 1, 1, 1, 0), (0, 2, 1, 1, 0), or (0, 1, 2, 1, 0) in this order when d is 2). Here, a, b, c, d, e, f, g, h, i, j, and k indicate the numbers of repetitions of sequences. For example, (YX) a indicates a sequence consisting of 4 nucleotides of YXYX when a=2; and indicates a sequence consisting of 6 nucleotides of YXYXYX when a=3; and there is no sequence in parentheses when a=0.

The double-stranded siRNA is cleaved by Ago2. In some embodiments, in the case of the 21-nucleotide long chemically modified siRNA of the present disclosure, the site of cleavage by Ago2 is the bond between positions 9 and 10 or positions 10 and 11 from the 5′-end of sense strand.

In some embodiments, the chemically modified siRNA of the present disclosure is selected from the group consisting of the double-stranded RNA consisting of the sense strand of SEQ ID NO: 5 and the antisense strand of SEQ ID NO: 6, the double-stranded RNA consisting of the sense strand of SEQ ID NO: 13 and the antisense strand of SEQ ID NO: 14, the double-stranded RNA consisting of the sense strand of SEQ ID NO: 15 and the antisense strand of SEQ ID NO: 16, the double-stranded RNA consisting of the sense strand of SEQ ID NO: 17 and the antisense strand of SEQ ID NO: 18, the double-stranded RNA consisting of the sense strand of SEQ ID NO: 19 and the antisense strand of SEQ ID NO: 20, the double-stranded RNA consisting of the sense strand of SEQ ID NO: 21 and the antisense strand of SEQ ID NO: 22, the double-stranded RNA consisting of the sense strand of SEQ ID NO: 23 and the antisense strand of SEQ ID NO: 24, the double-stranded RNA consisting of the sense strand of SEQ ID NO: 35 and the antisense strand of SEQ ID NO: 36, the double-stranded RNA consisting of the sense strand of SEQ ID NO: 37 and the antisense strand of SEQ ID NO: 38, the double-stranded RNA consisting of the sense strand of SEQ ID NO: 39 and the antisense strand of SEQ ID NO: 40, the double-stranded RNA consisting of the sense strand of SEQ ID NO: 41 and the antisense strand of SEQ ID NO: 42, the double-stranded RNA consisting of the sense strand of SEQ ID NO: 43 and the antisense strand of SEQ ID NO: 44, the double-stranded RNA consisting of the sense strand of SEQ ID NO: 45 and the antisense strand of SEQ ID NO: 46, the double-stranded RNA consisting of the sense strand of SEQ ID NO: 47 and the antisense strand of SEQ ID NO: 48, the double-stranded RNA consisting of the sense strand of SEQ ID NO: 49 and the antisense strand of SEQ ID NO: 50, the double-stranded RNA consisting of the sense strand of SEQ ID NO: 51 and the antisense strand of SEQ ID NO: 52, the double-stranded RNA consisting of the sense strand of SEQ ID NO: 53 and the antisense strand of SEQ ID NO: 54, the double-stranded RNA consisting of the sense strand of SEQ ID NO: 55 and the antisense strand of SEQ ID NO: 56, the double-stranded RNA consisting of the sense strand of SEQ ID NO: 57 and the antisense strand of SEQ ID NO: 58, the double-stranded RNA consisting of the sense strand of SEQ ID NO: 59 and the antisense strand of SEQ ID NO: 60, the double-stranded RNA consisting of the sense strand of SEQ ID NO: 63 and the antisense strand of SEQ ID NO: 64, the double-stranded RNA consisting of the sense strand of SEQ ID NO: 67 and the antisense strand of SEQ ID NO: 68, the double-stranded RNA consisting of the sense strand of SEQ ID NO: 71 and the antisense strand of SEQ ID NO: 72, the double-stranded RNA consisting of the sense strand of SEQ ID NO: 73 and the antisense strand of SEQ ID NO: 74, the double-stranded RNA consisting of the sense strand of SEQ ID NO: 75 and the antisense strand of SEQ ID NO: 76, the double-stranded RNA consisting of the sense strand of SEQ ID NO: 79 and the antisense strand of SEQ ID NO: 80, the double-stranded RNA consisting of the sense strand of SEQ ID NO: 81 and the antisense strand of SEQ ID NO: 82, the double-stranded RNA consisting of the sense strand of SEQ ID NO: 85 and the antisense strand of SEQ ID NO: 86, the double-stranded RNA consisting of the sense strand of SEQ ID NO: 87 and the antisense strand of SEQ ID NO: 88, the double-stranded RNA consisting of the sense strand of SEQ ID NO: 89 and the antisense strand of SEQ ID NO: 90, the double-stranded RNA consisting of the sense strand of SEQ ID NO: 91 and the antisense strand of SEQ ID NO: 92, the double-stranded RNA consisting of the sense strand of SEQ ID NO: 93 and the antisense strand of SEQ ID NO: 94, the double-stranded RNA consisting of the sense strand of SEQ ID NO: 97 and the antisense strand of SEQ ID NO: 98, the double-stranded RNA consisting of the sense strand of SEQ ID NO: 99 and the antisense strand of SEQ ID NO: 100, the double-stranded RNA consisting of the sense strand of SEQ ID NO: 103 and the antisense strand of SEQ ID NO: 104, the double-stranded RNA consisting of the sense strand of SEQ ID NO: 105 and the antisense strand of SEQ ID NO: 106, the double-stranded RNA consisting of the sense strand of SEQ ID NO: 107 and the antisense strand of SEQ ID NO: 108, the double-stranded RNA consisting of the sense strand of SEQ ID NO: 109 and the antisense strand of SEQ ID NO: 110, the double-stranded RNA consisting of the sense strand of SEQ ID NO: 111 and the antisense strand of SEQ ID NO: 112, the double-stranded RNA consisting of the sense strand of SEQ ID NO: 113 and the antisense strand of SEQ ID NO: 114, the double-stranded RNA consisting of the sense strand of SEQ ID NO: 117 and the antisense strand of SEQ ID NO: 118, the double-stranded RNA consisting of the sense strand of SEQ ID NO: 119 and the antisense strand of SEQ ID NO: 120, the double-stranded RNA consisting of the sense strand of SEQ ID NO: 123 and the antisense strand of SEQ ID NO: 124, the double-stranded RNA consisting of the sense strand of SEQ ID NO: 125 and the antisense strand of SEQ ID NO: 126, the double-stranded RNA consisting of the sense strand of SEQ ID NO: 127 and the antisense strand of SEQ ID NO: 128, and the double-stranded RNA consisting of the sense strand of SEQ ID NO: 129 and the antisense strand of SEQ ID NO: 130, described in Table 1 below.

In some embodiments, the chemically modified siRNA of the present disclosure is selected from the group consisting of the double-stranded RNA consisting of the sense strand of SEQ ID NO: 13 and the antisense strand of SEQ ID NO: 14, the double-stranded RNA consisting of the sense strand of SEQ ID NO: 21 and the antisense strand of SEQ ID NO: 22, the double-stranded RNA consisting of the sense strand of SEQ ID NO: 39 and the antisense strand of SEQ ID NO: 40, the double-stranded RNA consisting of the sense strand of SEQ ID NO: 43 and the antisense strand of SEQ ID NO: 44, the double-stranded RNA consisting of the sense strand of SEQ ID NO: 49 and the antisense strand of SEQ ID NO: 50, the double-stranded RNA consisting of the sense strand of SEQ ID NO: 53 and the antisense strand of SEQ ID NO: 54, the double-stranded RNA consisting of the sense strand of SEQ ID NO: 55 and the antisense strand of SEQ ID NO: 56, the double-stranded RNA consisting of the sense strand of SEQ ID NO: 57 and the antisense strand of SEQ ID NO: 58, the double-stranded RNA consisting of the sense strand of SEQ ID NO: 59 and the antisense strand of SEQ ID NO: 60, the double-stranded RNA consisting of the sense strand of SEQ ID NO: 87 and the antisense strand of SEQ ID NO: 88, the double-stranded RNA consisting of the sense strand of SEQ ID NO: 89 and the antisense strand of SEQ ID NO: 90, the double-stranded RNA consisting of the sense strand of SEQ ID NO: 91 and the antisense strand of SEQ ID NO: 92, the double-stranded RNA consisting of the sense strand of SEQ ID NO: 93 and the antisense strand of SEQ ID NO: 94, the double-stranded RNA consisting of the sense strand of SEQ ID NO: 111 and the antisense strand of SEQ ID NO: 112, the double-stranded RNA consisting of the sense strand of SEQ ID NO: 113 and the antisense strand of SEQ ID NO: 114, the double-stranded RNA consisting of the sense strand of SEQ ID NO: 123 and the antisense strand of SEQ ID NO: 124, the double-stranded RNA consisting of the sense strand of SEQ ID NO: 125 and the antisense strand of SEQ ID NO: 126, the double-stranded RNA consisting of the sense strand of SEQ ID NO: 127 and the antisense strand of SEQ ID NO: 128, and the double-stranded RNA consisting of the sense strand of SEQ ID NO: 129 and the antisense strand of SEQ ID NO: 130, described in Table 1 below.

In some embodiments, the chemically modified siRNA of the present disclosure is selected from the group consisting of the double-stranded RNA consisting of the sense strand of SEQ ID NO: 3 and the antisense strand of SEQ ID NO: 4, the double-stranded RNA consisting of the sense strand of SEQ ID NO: 7 and the antisense strand of SEQ ID NO: 8, the double-stranded RNA consisting of the sense strand of SEQ ID NO: 9 and the antisense strand of SEQ ID NO: 10, the double-stranded RNA consisting of the sense strand of SEQ ID NO: 11 and the antisense strand of SEQ ID NO: 12, the double-stranded RNA consisting of the sense strand of SEQ ID NO: 25 and the antisense strand of SEQ ID NO: 26, the double-stranded RNA consisting of the sense strand of SEQ ID NO: 27 and the antisense strand of SEQ ID NO: 28, the double-stranded RNA consisting of the sense strand of SEQ ID NO: 29 and the antisense strand of SEQ ID NO: 30, the double-stranded RNA consisting of the sense strand of SEQ ID NO: 31 and the antisense strand of SEQ ID NO: 32, the double-stranded RNA consisting of the sense strand of SEQ ID NO: 33 and the antisense strand of SEQ ID NO: 34, the double-stranded RNA consisting of the sense strand of SEQ ID NO: 65 and the antisense strand of SEQ ID NO: 66, the double-stranded RNA consisting of the sense strand of SEQ ID NO: 83 and the antisense strand of SEQ ID NO: 84, the double-stranded RNA consisting of the sense strand of SEQ ID NO: 121 and the antisense strand of SEQ ID NO: 122, described in Table 1 below.

TABLE 1
Strands (S = sense strands, AS = antisense strands)

| | | | SEQ ID |
|---|---|---|---|
| siRNA | Strand | Sequence (5′→3′) | NO |
| siRNA-010 | S | GGGAGAGGCUGUAUUAAUUdTdT | 1 |
| | AS | AAUUAAUACAGCCUCUCCCdTdT | 2 |
| siRNA-010-2 | S | GfsgsGfaGfaGfgCfuGfuAfuUfaAfuUfsusUf | 3 |
| | AS | asAfsuUfaAfuAfcAfgCfcUfcUfcCfcsUfsu | 4 |
| siRNA-010-4 | S | GfsgsGfaGfaGfgCfUfgUfaUfuAfaUfusUfsu | 5 |
| | AS | AfsasUfuAfaUfaCfagCfcUfcUfcCfcsUfsu | 6 |
| siRNA-010-5 | S | GfsgsGfaGfaGfgCfugUfaUfuAfaUfusUfsu | 7 |
| | AS | AfsasUfuAfaUfaCfAfgCfcUfcUfcCfcsUfsu | 8 |
| siRNA-010-12 | S | GfsgsGfaGfaGfgCfUfGfuAfuUfaAfuUfsusUf | 9 |
| | AS | asAfsuUfaAfuAfcagCfcUfcUfcCfcsUfsu | 10 |
| siRNA-010-kh | S | gsGfsgAfgAfgGfCfuGfUfaUfuAfaUfusUfsu | 11 |
| | AS | AfsasUfuAfaUfacAfgcCfuCfuCfcCfsusUf | 12 |
| siRNA-010-8 | S | GfsgsGfaGfaGfgCfUfguAfuUfaAfuUfsusUf | 13 |
| | AS | asAfsuUfaAfuAfCfagCfcUfcUfcCfcsUfsu | 14 |
| siRNA-010-4-3 | S | GfsgsGfaGfaGfgCfUfgUfaUfuAfauUfsusUf | 15 |
| | AS | asAfsUfuAfaUfaCfagCfcUfcUfcCfcsUfsu | 16 |
| siRNA-010-4-4 | S | gsGfsgAfgAfggCfUfgUfaUfuAfaUfusUfsu | 17 |
| | AS | AfsasUfuAfaUfaCfagCfCfuCfuCfcCfsusUf | 18 |
| siRNA-010-4-7 | S | gsgsGfaGfaGfgCfUfgUfaUfuAfaUfusUfsu | 19 |
| | AS | AfsasUfuAfaUfaCfagCfcUfcUfcCfCfsusUf | 20 |
| siRNA-010-4-13 | S | GfsgsGfaGfaGfgCfUfgUfaUfuaauUfsusUf | 21 |
| | AS | asAfsUfUfAfaUfaCfagCfcUfcUfcCfcsUfsu | 22 |
| siRNA-010-4-8 | S | gsGfsgAfgaggCfUfgUfaUfuAfaUfusUfsu | 23 |
| | AS | AfsasUfuAfaUfaCfagCfCfUfCfuCfcCfsusUf | 24 |
| siRNA-010-4-14 | S | GfsgsGfaGfaGfgCfUfgUfaUfuAfaUfusUfsu | 25 |
| | AS | AfsasUfuAfaUfaCfALgCfcUfcUfcCfcsUfsu | 26 |
| siRNA-010-4-15 | S | GfsgsGfaGfaGfgCfTLgUfaUfuAfaUfusUfsu | 27 |
| | AS | AfsasUfuAfaUfaCfagCfcUfcUfcCfcsUfsu | 28 |
| siRNA-010-4-16 | S | GfsgsGfaGfaGfgCfUfgUfaUfuAfaUfusUfsu | 29 |
| | AS | AfsasUfuAfaUfaCfALGLCfcUfcUfcCfcsUfsu | 30 |
| siRNA-010-4-18 | S | GfsgsGfaGfaGfgdCdTgUfaUfuAfaUfusUfsu | 31 |
| | AS | AfsasUfuAfaUfaCfagCfcUfcUfcCfcsUfsu | 32 |
| siRNA-010-4-19 | S | dGsgsdGadGadGgCfUfgdTadTudAadTusdTsu | 33 |
| | AS | dAsasdTudAadTadCagdCcdTcdTcdCesdTsu | 34 |
| siRNA-010-4-23 | S | gsGfsgAfgAfggCfUfgUfaUfuaauUfsusUf | 35 |
| | AS | asAfsUfUfAfaUfaCfagCfCfuCfuCfcCfsusUf | 36 |
| siRNA-010-4-24 | S | gsGfsgAfgaggCfUfgUfaUfuaauUfsusUf | 37 |
| | AS | asAfsUfUfAfaUfaCfagCfCfUfCfuCfcCfsusUf | 38 |
| siRNA-010-16 | S | GfsgsGfaGfaGfgCfUfGfUfaUfuAfaUfusUfsu | 39 |
| | AS | AfsasUfuAfaUfacagCfcUfcUfcCfcsUfsu | 40 |
| siRNA-010-16-2 | S | gsGfsgAfgaGfgCfUfGfUfaUfuAfaUfusUfsu | 41 |
| | AS | AfsasUfuAfaUfacagCfcUfCfuCfcCfsusUf | 42 |
| siRNA-010-16-6 | S | GfsgsGfaGfaGfgCfUfGfUfaUfuaAfuUfsusUf | 43 |
| | AS | asAfsuUfAfaUfacagCfcUfcUfcCfcsUfsu | 44 |
| siRNA-010-16-7 | S | GfsgsGfaGfaGfgCfUfGfUfaUfuAfauUfsusUf | 45 |
| | AS | asAfsUfuAfaUfacagCfcUfcUfcCfcsUfsu | 46 |
| siRNA-010-16-11 | S | GfsgsGfaGfaGfgCfUfGfUfauuaAfuUfsusUf | 47 |
| | AS | asAfsuUfAfAfUfacagCfcUfcUfcCfcsUfsu | 48 |
| siRNA-010-16-12 | S | GfsgsGfaGfaGfgCfUfGfUfaUfuaauUfsusUf | 49 |
| | AS | asAfsUfUfAfaUfacagCfcUfcUfcCfcsUfsu | 50 |
| siRNA-010-16-9 | S | gsGfsgagaGfgCfUfGfUfaUfuAfaUfusUfsu | 51 |
| | AS | AfsasUfuAfaUfacagCfcUfCfUfCfcCfsusUf | 52 |
| siRNA-010-16-13 | S | gsGfsgagaGfgCfUfGfUfaUfuaauUfsusUf | 53 |
| | AS | asAfsUfUfAfaUfacagCfcUfCfUfCfcCfsusUf | 54 |
| siRNA-010-16-14 | S | gsGfsgAfgaGfgCfUfGfUfaUfuaauUfsusUf | 55 |
| | AS | asAfsUfUfAfaUfacagCfcUfCfuCfcCfsusUf | 56 |
| siRNA-010-16-15 | S | gsGfsgagaGfgCfUfGfUfaUfuaAfuUfsusUf | 57 |
| | AS | asAfsuUfAfaUfacagCfcUfCfUfCfcCfsusUf | 58 |
| siRNA-010-16-16 | S | gsGfsgAfgaGfgCfUfGfUfaUfuaAfuUfsusUf | 59 |
| | AS | asAfsuUfAfaUfacagCfcUfCfuCfcCfsusUf | 60 |
| siRNA-002 | S | GGAUCGCAGGGAGAGGCUGdTdT | 61 |
| | AS | CAGCCUCUCCCUGCGAUCCdTdT | 62 |
| siRNA-002-4 | S | GfsgsAfuCfgCfaGfGfgAfgAfgGfcUfgsUfsu | 63 |
| | AS | CfsasGfcCfuCfuCfccUfgCfgAfuCfcsUfsu | 64 |
| siRNA-002-12 | S | GfsgsAfuCfgCfaGfGfGfaGfaGfgCfuGfsusUf | 65 |
| | AS | csAfsgCfcUfcUfcccUfgCfgAfuCfcsUfsu | 66 |
| siRNA-002-16 | S | GfsgsAfuCfgCfaGfGfGfAfgAfgGfcUfgsUfsu | 67 |
| | AS | CfsasGfcCfuCfucccUfgCfgAfuCfcsUfsu | 68 |
| siRNA-003 | S | GAUCGCAGGGAGAGGCUGUdTdT | 69 |
| | AS | ACAGCCUCUCCCUGCGAUCdTdT | 70 |
| siRNA-003-4 | S | GfsasUfcGfcAfgGfGfaGfaGfgCfuGfusUfsu | 71 |
| | AS | AfscsAfgCfcUfcUfccCfuGfcGfaUfcsUfsu | 72 |
| siRNA-003-16 | S | GfsasUfcGfcAfgGfGfAfGfaGfgCfuGfusUfsu | 73 |
| | AS | AfscsAfgCfcUfcuccCfuGfcGfaUfcsUfsu | 74 |
| siRNA-003-16-13 | S | gsAfsucgcAfgGfGfAfGfaGfgcugUfsusUf | 75 |
| | AS | asCfsAfGfCfcUfcuccCfuGfCfGfAfuCfsusUf | 76 |
| siRNA-006 | S | CGCAGGGAGAGGCUGUAUUdTdT | 77 |
| | AS | AAUACAGCCUCUCCCUGCGdTdT | 78 |
| siRNA-006-4 | S | CfsgsCfaGfgGfaGfAfgGfcUfgUfaUfusUfsu | 79 |
| | AS | AfsasUfaCfaGfcCfucUfcCfcUfgCfgsUfsu | 80 |
| siRNA-006-8 | S | CfsgsCfaGfgGfaGfAfggCfuGfuAfuUfsusUf | 81 |
| | AS | asAfsuAfcAfgCfCfucUfcCfcUfgCfgsUfsu | 82 |
| siRNA-006-12 | S | CfsgsCfaGfgGfaGfAfGfgCfuGfuAfuUfsusUf | 83 |
| | AS | asAfsuAfcAfgCfcucUfcCfcUfgCfgsUfsu | 84 |
| siRNA-006-16 | S | CfsgsCfaGfgGfaGfAfGfGfcUfgUfaUfusUfsu | 85 |
| | AS | AfsasUfaCfaGfccucUfcCfcUfgCfgsUfsu | 86 |
| siRNA-006-16-13 | S | csGfscaggGfaGfAfGfGfcUfguauUfsusUf | 87 |
| | AS | asAfsUfAfCfaGfccucUfcCfCfUfGfcGfsusUf | 88 |
| siRNA-006-16-14 | S | csGfscAfggGfaGfAfGfGfcUfguauUfsusUf | 89 |
| | AS | asAfsUfAfCfaGfccucUfcCfCfuGfcGfsusUf | 90 |
| siRNA-006-16-15 | S | csGfscaggGfaGfAfGfGfcUfguAfuUfsusUf | 91 |
| | AS | asAfsuAfCfaGfccucUfcCfCfUfGfcGfsusUf | 92 |
| siRNA-006-16-16 | S | csGfscAfggGfaGfAfGfGfcUfguAfuUfsusUf | 93 |
| | AS | asAfsuAfCfaGfccucUfcCfCfuGfcGfsusUf | 94 |
| siRNA-008 | S | CAGGGAGAGGCUGUAUUAAdTdT | 95 |
| | AS | UUAAUACAGCCUCUCCCUGdTdT | 96 |
| siRNA-008-16 | S | CfsasGfgGfaGfaGfGfCfUfgUfaUfuAfasUfsu | 97 |
| | AS | UfsusAfaUfaCfagccUfcUfcCfcUfgsUfsu | 98 |
| siRNA-008-16-16 | S | csAfsgGfgaGfaGfGfCfUfgUfauUfaAfsusUf | 99 |
| | AS | usUfsaAfUfaCfagccUfcUfCfcCfuGfsusUf | 100 |
| siRNA-009 | S | AGGGAGAGGCUGUAUUAAUdTdT | 101 |
| | AS | AUUAAUACAGCCUCUCCCUdTdT | 102 |
| siRNA-009-4 | S | AfsgsGfgAfgAfgGfCfuGfuAfuUfaAfusUfsu | 103 |
| | AS | AfsusUfaAfuAfcAfgcCfuCfuCfcCfusUfsu | 104 |
| siRNA-009-16 | S | AfsgsGfgAfgAfgGfCfUfGfuAfuUfaAfusUfsu | 105 |
| | AS | AfsusUfaAfuAfcagcCfuCfuCfcCfusUfsu | 106 |
| siRNA-009-16-13 | S | asGfsggagAfgGfCfUfGfuAfuuaaUfsusUf | 107 |
| | AS | asUfsUfAfAfuAfcagcCfuCfUfCfCfcUfsusUf | 108 |
| siRNA-009-16-14 | S | asGfsgGfagAfgGfCfUfGfuAfuuaaUfsusUf | 109 |
| | AS | asUfsUfAfAfuAfcagcCfuCfUfcCfcUfsusUf | 110 |
| siRNA-009-16-15 | S | asGfsggagAfgGfCfUfGfuAfuuAfaUfsusUf | 111 |
| | AS | asUfsuAfAfuAfcagcCfuCfUfCfCfcUfsusUf | 112 |
| siRNA-009-16-16 | S | asGfsgGfagAfgGfCfUfGfuAfuuAfaUfsusUf | 113 |
| | AS | asUfsuAfAfuAfcagcCfuCfUfcCfcUfsusUf | 114 |
| siRNA-011 | S | GGAGAGGCUGUAUUAAUUAdTdT | 115 |
| | AS | UAAUUAAUACAGCCUCUCCdTdT | 116 |
| siRNA-011-4 | S | GfsgsAfgAfgGfcUfGfuAfuUfaAfuUfasUfsu | 117 |
| | AS | UfsasAfuUfaAfuAfcaGfcCfuCfuCfcsUfsu | 118 |
| siRNA-011-8 | S | GfsgsAfgAfgGfcUfGfuaUfuAfaUfuAfsusUf | 119 |
| | AS | usAfsaUfuAfaUfAfcaGfcCfuCfuCfcsUfsu | 120 |
| siRNA-011-12 | S | GfsgsAfgAfgGfcUfGfUfaUfuAfaUfuAfsusUf | 121 |
| | AS | usAfsaUfuAfaUfacaGfcCfuCfuCfcsUfsu | 122 |
| siRNA-011-16-13 | S | gsGfsagagGfcUfGfUfAfuUfaauuAfsusUf | 123 |
| | AS | usAfsAfUfUfaAfuacaGfcCfUfCfUfcCfsusUf | 124 |
| siRNA-011-16-14 | S | gsGfsaGfagGfcUfGfUfAfuUfaauuAfsusUf | 125 |
| | AS | usAfsAfUfUfaAfuacaGfcCfUfcUfcCfsusUf | 126 |
| siRNA-011-16-15 | S | gsGfsagagGfcUfGfUfAfuUfaaUfuAfsusUf | 127 |
| | AS | usAfsaUfUfaAfuacaGfcCfUfCfUfcCfsusUf | 128 |
| siRNA-011-16-16 | S | gsGfsaGfagGfcUfGfUfAfuUfaaUfuAfsusUf | 129 |
| | AS | usAfsaUfUfaAfuacaGfcCfUfcUfcCfsusUf | 130 |

The siRNA-010, -002, -003, -006, -008, -009, and -011 listed in Table 1 are all naked siRNA, and were prepared and used in tests to compare their stability against RNases and RNAi activity with the chemically modified siRNAs of the present disclosure.

The chemically modified nucleotides contained in the chemically modified siRNAs listed in Table 1 and their abbreviations are shown in Table 2 below.

TABLE 2

| Abbreviation | Nucleotide |
|---|---|
| Af | 2′-F-adenosine |
| Cf | 2′-F-cytidine |
| Gf | 2′-F-guanosine |
| Uf | 2′-F-uridine |
| A | adenosine |
| C | cytidine |
| G | guanosine |
| U | uridine |
| a | 2′-OMe-adenosine |
| c | 2′-OMe-cytidine |
| g | 2′-OMe-guanosine |
| u | 2′-OMe-uridine |
| dA | 2′-deoxy-adenosine |
| dC | 2′-deoxy-cytidine |
| dG | 2′-deoxy-guanosine |
| dT | 2′-deoxy-thymidine |
| AL | LNA-modified adenosine |
| 5meCL | LNA-modified 5-methyl cytidine |
| GL | LNA-modified guanosine |
| TL | LNA-modified uridine |
| s | phosphorothioate bonds |

In Table 2, the term “LNA (locked nucleic acid)-modified” refers to a chemically modified nucleotide in which ribose constituting the nucleotide is bridged by methylene between a 2′-0 atom and a 4′-C atom.

In some embodiments, although the sense strands and antisense strands constituting the chemically modified siRNA of the present disclosure have the sequences listed in Table 1, they may also have substantially the same sequences as those listed therein. The term “substantially the same sequences” means that chemical modifications and mismatched bases may be included in the sequences described in Table 1, so long as the antisense strands of siRNA and target mRNA retain the ability to form double-stranded RNA. In some embodiments, the number of the mismatched bases is three or less. In some embodiments, the number of the mismatched bases may be up to one.

The sense strands and antisense strands constituting the chemically modified siRNA of the present disclosure may include overhang of dinucleotide at the 3-end. In some embodiments, the chemically modified siRNA of the present disclosure includes UU (U: uridine) as overhang.

Although siRNA typically has phosphodiester bonds, as for the chemically modified siRNA of the present disclosure, in the three nucleotides located at the 5′-end and 3′-end of both sense strands and antisense strands, the two phosphodiester bonds between adjacent nucleotides are replaced with phosphorothioate bonds.

The chemically modified siRNA of the present disclosure can be produced by one of the synthetic methods for nucleic acid molecules well known to those skilled in the art. Examples of the synthetic methods include those described in “Development and Application of Nucleic Acid Medicines” (CMC Publishing, 2016) and “Synthetic Techniques for Peptides, Nucleic Acids, and Carbohydrate Chains Contributing to Development of Medium-Molecule Medicines” (CMC Publishing, 2018).

The chemically modified siRNA of the present disclosure can be made double-stranded by associating a synthesized single-stranded oligonucleotide with another complementary single-stranded oligonucleotide. Specific examples of the method for the association include a method of annealing the complementary oligonucleotides to each other by heating double-stranded oligonucleotides to a temperature of dissociation, and then cooling down gradually.

The oligonucleotide can be synthesized by a solid-phase synthesis using commercially available amidite. The solid-phase synthesis is performed using a commercially available nucleic acid synthesizer and a solid-phase carrier. The 3′-end of the monomer nucleotide is bonded to the surface of the solid-phase carrier via an alkyl strand, and amidite is added thereto. That is, the desired oligonucleotide can be synthesized by repeating a cycle of extending one nucleotide at a time from the 3′-end to the 5′-end of its sequence. The desired single-stranded RNA can be prepared by excising the oligonucleotide from the solid-phase carrier after the completion of the synthetic cycle, and deprotecting the base moiety and the 2′-position. However, in the cases of synthesizing 2′-F modified RNA, 2′-OMe modified RNA, or RNA in which the 2′-0 atom and the 4′-C atom are bridged with methylene, the step of deprotection is not necessary.

The chemically modified siRNA of the present disclosure can be synthesized by AJINOMOTO BIOPHARMA SERVICE GENE DESIGN Co., Ltd. using the phosphoroamidite method described above. The quality of the obtained siRNA can be confirmed by mass spectrometry and electrophoresis after simple purification by column.

The chemically modified siRNA of the present disclosure can be prepared by selecting a consecutive base sequence, being the target of FUS P525L mutation mRNA. Specifically, it is selected from an mRNA sequence of 19 to 21 nucleotides in the region containing the moiety of P525L mutation. If the sequence of the obtained siRNA can induce RNA interference and degrade FUS P525L mutation mRNA, a base sequence in which one or several nucleotides are substituted, deleted, inserted and/or added can be selected and prepared.

In some embodiments, a chemically modified siRNA or a salt thereof consisting of a sense strand and an antisense strand, wherein the antisense strand comprises a region complementary or substantially complementary to a part of mRNA encoding FUS P525L mutation protein, the complementary region is 19 to 21 nucleotides in length, the siRNA comprises at least one substitution selected from the group consisting of 2′-F-nucleotides, 2′-OMe-nucleotides, nucleotides in which a 2′-O atom and a 4′-C atom are bridged with methylene, 2′-deoxy-nucleotides, and phosphorothioate bonds that form internucleotide linkage can be produced by one of the synthetic methods of nucleic acid molecules well known to those skilled in the art.

In some embodiments, the chemically modified siRNA or a salt thereof comprising a motif of two or four consecutive 2′-F-nucleotides or 2′-OMe-nucleotides at or adjacent to the site of cleavage of the RNA strand by Ago2, and comprising 2′-F-nucleotides and 2′-OMe-nucleotides alternately along the RNA strand except the motif can be produced by one of the synthetic methods of nucleic acid molecules well known to those skilled in the art.

In some embodiments, the chemically modified siRNA or a salt thereof, wherein the siRNA is represented by formula (I) below:

(I)
Sense strand:
5′-(YX)a-(YY)b-(XY)c-(XX)d-(YX)e-(YY)f-(XY)g-
(XYX)h-(YXY)i-3′
Antisense strand:
3′-(YX)j-(XY)k-(XY)a-(XX)b-(YX)c-(YY)d-(XY)e-
(XX)f-(YX)g-(Y)h-(X)i-5′

(wherein, X and Y are 2′-F-nucleotides and 2′-OMe-nucleotides, respectively; a, b, c, d, e, f, g, h, i, j, and k are each independently an integer of 0 to 4;
(a, b, c, j, k) are (0, 0, 4, 1, 0), (0, 1, 3, 0, 1), (0, 2, 2, 0, 1), (1, 1, 2, 0, 1), (2, 1, 1, 0, 1), (1, 2, 1, 0, 1), (2, 2, 0, 0, 1), or (3, 1, 0, 0, 1) in this order;
(e, f, g, h, i) are (4, 0, 0, 0, 1), (3, 1, 0, 1, 0), (2, 2, 0, 1, 0), (2, 1, 1, 1, 0), (1, 1, 2, 1, 0), (1, 2, 1, 1, 0), (0, 2, 2, 1, 0), or (0, 1, 3, 1, 0) in this order when d is 1;
(e, f, g, h, i) are (3, 0, 0, 0, 1), (2, 1, 0, 1, 0), (1, 2, 0, 1, 0), (1, 1, 1, 1, 0), (0, 2, 1, 1, 0), or (0, 1, 2, 1, 0) in this order when d is 2); and is 21 nucleotides in length, can be produced by one of the synthetic methods of nucleic acid molecules well known to those skilled in the art.

The chemically modified siRNA of the present disclosure can be produced, as appropriate, by a person skilled in the art based on the base sequences disclosed herein. Specifically, a double-stranded RNA can be produced on the basis of any base sequences of SEQ ID NOs: 1 to 130. If one nucleotide strand is identified, a person skilled in the art can easily understand the base sequence of the other complementary nucleotide strand. The chemically modified siRNA of the present disclosure may be produced by using a commercially available nucleic acid synthesizer or the like, or may be obtained by utilizing a general synthesis contract service.

The chemically modified siRNA of the present disclosure can induce RNA interference, degrades FUS P525L mutation mRNA as a target, and selectively exhibits silencing of FUS P525L mutation involved in the onset of ALS.

The chemically modified siRNA of the present disclosure inhibits the expression of FUS P525L mutation. Meanwhile, it does not substantially affect the expression of wild-type FUS. That is, the chemically modified siRNA of the present disclosure selectively exhibits silencing of FUS P525L mutation without substantially silencing of wild-type FUS. The term “without substantially silencing of wild-type FUS” means that undesirable symptoms caused by the silencing of wild-type FUS in ALS do not substantially appear.

The silencing effect of the chemically modified siRNA of the present disclosure on the expression of FUS P525L mutation can be expressed as expression silencing rate (%) by the following calculation formula. In some embodiments, the silencing rate of the FUS P525L mutation by the chemically modified siRNA of the present disclosure is 30% or more. In some embodiments, the expression inhibition rate of the FUS P525L mutation by the chemically modified siRNA of the present disclosure is 50% or more.

Regarding the silencing effect of the chemically modified siRNA of the present disclosure on FUS expression, the selectivity for the FUS P525L mutation compared to the

$$Selectivity in expression rate = expression rate of wild - type FUS / expression rate of FUS P 525 L mutation ; or$$

$$Selectivity in silencing rate = silencing rate of FUS P 525 L mutation ( \% ) - silencing rate of wild - type FUS ( \% ) .$$

For both the expression rate and the silencing rate, the higher the numerical value, the greater the selectivity for silencing of FUS P525L mutation. The expression rate and silencing rate indicated by the siRNA of the present disclosure with respect to wild-type FUS and FUS P525L mutation are obtained by following calculation formula.

In some embodiments, the selectivity represented by the expression rate of the chemically modified siRNA of the present disclosure is 1.5 or more. In some embodiments, the selectivity represented by the expression rate of the chemically modified siRNA of the present disclosure is 2 or more. In some embodiments, the selectivity represented by the silencing rate of the chemically modified siRNA of the present disclosure is 20% or more. In some embodiments, the selectivity represented by the silencing rate of the chemically modified siRNA of the present disclosure is 40% or more. The selectivity of the FUS silencing by the chemically modified siRNA may be evaluated based on either of the selectivity represented by the expression rate or the selectivity represented by the silencing rate, or may be evaluated based on a combination of the both.

The chemically modified siRNA of the present disclosure may be in the form of a salt. In some embodiments, the salt is a pharmaceutically acceptable salt. In some embodiments, the salt includes, but is not limited to, an alkali metal salt such as sodium salt, potassium salt, lithium salt, and an alkaline earth metal salt such as calcium salt and magnesium salt.

The chemically modified siRNA or a salt thereof of the present disclosure is useful as a therapeutic agent for ALS, and its therapeutic effect can be evaluated, for example, by using the methods described in the following documents or a method equivalent thereto.

McCampbell A, et al. Antisense oligonucleotides extend survival and reverse decrement in muscle response in ALS models. J Clin Invest, 2018; 128: 3558-3567.

Akiyama T, et al. Aberrant axon branching via Fos-B dysregulation in FUS-ALS motor neurons. EBioMedicine, 2019; 45: 362-378.

Shiihashi G, Mislocated FUS is sufficient for gain-of-toxic-function amyotrophic lateral sclerosis phenotypes in mice. Brain, 2016; 139: 2380-94.

In some embodiments, a pharmaceutical composition for preventing or treating ALS comprising the chemically modified siRNA or a salt thereof of the present disclosure and a pharmaceutically acceptable carrier is provided. In some embodiments, the ALS is ALS having FUS P525L mutation.

In some embodiments, the pharmaceutical composition of the present disclosure may be in a dosage form for oral or parenteral use. These dosage forms can be formulated by a person skilled in the art, by appropriately combining pharmaceutically acceptable carriers and excipients, and mixing them in a unit dose form required for the generally accepted pharmaceutical practice. In some embodiments, the pharmaceutical composition of the present disclosure can be produced according to known methods, such as those described in the Japanese Pharmacopoeia or the United States Pharmacopoeia (USP).

In some embodiments, a method for preventing or treating ALS or ALS having FUS P525L mutation, comprising administering to a patient in need of treatment an effective amount of an expression inhibitor of FUS P525L mutation, which contains the chemically modified siRNA or a salt thereof of the present disclosure is provided.

In some embodiments, an expression inhibitor of FUS P525L mutation which contains the chemically modified siRNA or a salt thereof of the present disclosure, for the prevention or treatment of ALS or ALS having FUS P525L mutation is provided.

In some embodiments, an expression inhibitor of FUS P525L mutation which contains the chemically modified siRNA or a salt thereof of the present disclosure, for producing a prophylactic or therapeutic agent for ALS or ALS having FUS P525L mutation is provided.

In some embodiments, a method for preventing or treating ALS or ALS having FUS P525L mutation, comprising administering to a patient in need of treatment an effective amount of an expression inhibitor of FUS P525L mutation, which contains the chemically modified siRNA or a salt thereof of the present disclosure and a pharmaceutically acceptable carrier is provided.

In some embodiments, a pharmaceutical composition comprising the chemically modified siRNA or a salt thereof of the present disclosure and a pharmaceutically acceptable carrier, for the prevention or treatment of ALS or ALS having FUS P525L mutation is provided.

In some embodiments, a pharmaceutical composition comprising the chemically modified siRNA or a salt thereof of the present disclosure and a pharmaceutically acceptable carrier, for producing a prophylactic or therapeutic agent for ALS or ALS having FUS P525L mutation is provided.

The present invention will be described with reference to examples. However, the present invention is not limited to the following examples.

### Example 1

Synthesis of cDNA of Human FUS_{wild-type} and FUS_{P525L}

The cDNA sequence of human wild-type FUS (hereinafter referred to as FUS_{wild-type}) (sequence 1) is shown in SEQ ID NO: 131, and the cDNA sequence of human FUS P525L mutation (hereinafter referred to as FUS_{P525L}) (sequence 2) is shown in SEQ ID NO: 132. Artificial genes were obtained from a GenScript Japan Co. Ltd. Synthetic genes were inserted into the BamHI/XhoI site in the multi-cloning site of pcDNA3.1+ vector.

(Sequence 1)
(SEQ ID NO: 131)
tgcgcggacatggcctcaaacgattatacccaacaagcaacccaaagctatggggcctaccccacccagcccgggcagggctattc
ccagcagagcagtcagccctacggacagcagagttacagtggttatagccagtccacggacacttcaggctatggccagagcagct
attcttcttatggccagagccagaacagctatggaactcagtcaactccccagggatatggctcgactggcggctatggcagtagcca
gagctcccaatcgtcttacgggcagcagtcctcctatcctggctatggccagcagccagctcccagcagcacctcgggaagttacggt
agcagttctcagagcagcagctatgggcagccccagagtgggagctacagccagcagcctagctatggtggacagcagcaaagct
atggacagcagcaaagctataatccccctcagggctatggacagcagaaccagtacaacagcagcagtggtggtggaggtggaggt
ggaggtggaggtaactatggccaagatcaatcctccatgagtagtggtggtggcagtggtggcggttatggcaatcaagaccagagt
ggtggaggtggcagcggtggctatggacagcaggaccgtggaggccgcggcaggggggcagtggtggcggcggcggcggcg
gcggtggtggttacaaccgcagcagtggtggctatgaacccagaggtcgtggaggtggccgtggaggcagaggtggcatgggcg
gaagtgaccgtggtggcttcaataaatttggtggccctcgggaccaaggatcacgtcatgactccgaacaggataattcagacaacaa
caccatctttgtgcaaggcctgggtgagaatgttacaattgagtctgtggctgattacttcaagcagattggtattattaagacaaacaaga
aaacgggacagcccatgattaatttgtacacagacagggaaactggcaagctgaagggagaggcaacggtctcttttgatgacccac
cttcagctaaagcagctattgactggtttgatggtaaagaattctccggaaatcctatcaaggtctcatttgctactcgccgggcagacttt
aatcggggtggtggcaatggtcgtggaggccgagggcgaggaggacccatgggccgtggaggctatggaggtggtggcagtggt
ggtggtggccgaggaggatttcccagtggaggtggtggcggtggaggacagcagcgagctggtgactggaagtgtcctaatcccac
ctgtgagaatatgaacttctcttggaggaatgaatgcaaccagtgtaaggcccctaaaccagatggcccaggagggggaccaggtgg
ctctcacatggggggtaactacggggatgatcgtcgtggtggcagaggaggctatgatcgaggcggctaccggggccgcggcggg
gaccgtggaggcttccgagggggccggggtggtggggacagaggtggctttggccctggcaagatggattccaggggtgagcac
agacaggatcgcagggagaggccgtattaattagcctggctccccaggttctggaacagctttttgtcctgtacccagtgttaccctcgtt
attttgtaaccttccaattcctgatcacccaagggtttttttgtgtcggactatgtaattgtaactatacctctggttcccattaaaagtgacc
attttagttaaaaaaaa
(Sequence 2)
(SEQ ID NO: 132)
tgcgcggacatggcctcaaacgattatacccaacaagcaacccaaagctatggggcctaccccacccagcccgggcagggctattc
ccagcagagcagtcagccctacggacagcagagttacagtggttatagccagtccacggacacttcaggctatggccagagcagct
attcttcttatggccagagccagaacagctatggaactcagtcaactccccagggatatggctcgactggcggctatggcagtagcca
gagctcccaatcgtcttacgggcagcagtcctcctatcctggctatggccagcagccagctcccagcagcacctcgggaagttacggt
agcagttctcagagcagcagctatgggcagccccagagtgggagctacagccagcagcctagctatggtggacagcagcaaagct
atggacagcagcaaagctataatccccctcagggctatggacagcagaaccagtacaacagcagcagtggtggtggaggtggaggt
ggaggtggaggtaactatggccaagatcaatcctccatgagtagtggtggtggcagtggtggcggttatggcaatcaagaccagagt
ggtggaggtggcagcggtggctatggacagcaggaccgtggaggccgcggcaggggtggcagtggtggcggcggcggcggcg
gcggtggtggttacaaccgcagcagtggtggctatgaacccagaggtcgtggaggtggccgtggaggcagaggtggcatgggcg
gaagtgaccgtggtggcttcaataaatttggtggccctcgggaccaaggatcacgtcatgactccgaacaggataattcagacaacaa
caccatctttgtgcaaggcctgggtgagaatgttacaattgagtctgtggctgattacttcaagcagattggtattattaagacaaacaaga
aaacgggacagcccatgattaatttgtacacagacagggaaactggcaagctgaagggagaggcaacggtctcttttgatgacccac
cttcagctaaagcagctattgactggtttgatggtaaagaattctccggaaatcctatcaaggtctcatttgctactcgccgggcagacttt
aatcggggtggtggcaatggtcgtggaggccgagggcgaggaggacccatgggccgtggaggctatggaggtggtggcagtggt
ggtggtggccgaggaggatttcccagtggaggtggtggcggtggaggacagcagcgagctggtgactggaagtgtcctaatcccac
ctgtgagaatatgaacttctcttggaggaatgaatgcaaccagtgtaaggcccctaaaccagatggcccaggagggggaccaggtgg
ctctcacatggggggtaactacggggatgatcgtcgtggtggcagaggaggctatgatcgaggcggctaccggggccgcggcggg
gaccgtggaggcttccgagggggccggggtggtggggacagaggtggctttggccctggcaagatggattccaggggtgagcac
agacaggatcgcagggagaggctgtattaattagcctggctccccaggttctggaacagctttttgtcctgtacccagtgttaccctcgtt
attttgtaaccttccaattcctgatcacccaagggtttttttgtgtcggactatgtaattgtaactatacctctggttcccattaaaagtgacc
attttagttaaaaaaaa

### Example 2

Construction of Gene Expression Vector of GFP-Fused FUS_{wild-type} and FP635-Fused FUS_{P525L}

PCR was performed on pTurboGFP vector, pTurboFP635 vector, and the artificial gene prepared in Example 1 using the primer set shown in Table 3. The PCR was performed by mixing 25 μL of PrimeSTAR Max Premix (Takara Bio Inc.), 4 μL each of 2.5 μM Primer (final concentration 0.2 μM), 1 μL (20 ng) of template, and 20 μL of water, incubating at 98° C. for 10 seconds, followed by 35 temperature cycles of 98° C. for 10 seconds, 55° C. for 5 seconds, and 72° C. for 10 seconds (25 seconds if vector was used as template). The vector-amplified fragment and the FUS gene-amplified fragment were coupled by an In-Fusion reaction. That is, 2 μL of the insert fragment, 1 μL of the vector amplification product, 2 μL of 5× In-Fusion HD Enzyme Premix (Takara Bio Inc.), and 5 μL of water were mixed, reacted at 50° C. for 15 minutes, and transformed into NEB Turbo Competent E. coli (New England Biolabs Japan). Next, a plasmid vector was prepared from the transformant, it was confirmed that the cDNA of interest had been properly inserted from the DNA sequence. The FUS_{wild-type} and FUS_{P525L} were cloned in-frame into pTurboGFP vector (Evrogen) and pTurboFP635 vector (Evrogen), respectively, so that TurboGFP fluorescent protein was added to the N-terminal of FUS_{wild-type}, and TurboFP635 fluorescent protein was added to the N-terminal of FUS_{P525L}.

TABLE 3

| | | SEQ ID |
|---|---|---|
| Template | Primer Sequences | NO |
| pTurboGFP-c vector | Forward: | 133 |
| | TCTCGAGCTCAAGCTTCGAATTCTG | |
| | Reverse: | 134 |
| | TCTTTCTTCACCGGCATCTGCATCC | |
| pcDNA3.1(+)_FUS_{wild-type} | Forward: | 135 |
| | GCCGGTGAAGAAAGAATGGCCTCAAACG | |
| | ATTATAC | |
| | Reverse: | 136 |
| | AGCTTGAGCTCGAGATTTTTTTTAACTAA | |
| | AATGGTCAC | |
| pTurboFP635-c vector | Forward: | 137 |
| | TCTCGAGCTCAAGCTTCGAATTCTG | |
| | Reverse: | 138 |
| | TCTGAGTCCGGAGCTGTGCCCCAGT | |
| pcDNA3.1(+)_FUSP525L | Forward: | 139 |
| | AGCTCCGGACTCAGAATGGCCTCAAACG | |
| | ATTATAC | |
| | Reverse: | 140 |
| | AGCTTGAGCTCGAGATTTTTTTTAACTAA | |
| | AATGGTCAC | |

### Example 3

### Cell Culture

HEK293 cells were cultured at 37° C. and in an environment of 5% CO₂ using Advanced DMEM (Thermo Fischer Scientific Co. LTD.) containing 10% FBS and 4 mM GlutaMAX® Supplement. HEK293 cells were purchased from the JCRB cell bank at Culture Resources Laboratory of National Institutes of Biomedical Innovation, Health and Nutrition (cell number JCRB9068).

### Example 4

### Production of FUS Knockout (KO) HEK293 Cell Strains

FUS/TLS CRISPR/Cas9 KO (sc-400612) plasmid (Santa Cruz) and FUS/TLS HDR (sc-400612-HDR) plasmid (h) (Santa Cruz) were transfected using TransIT®-293 Transfection Reagent (Mirus) to produce FUS KO HEK cell strains. By confirming whether or not the FUS mRNA is expressed by RT-PCR (using SuperScript® IV One-Step RT-PCR System with ezDNase®, invitrogen, #12595100), the FUS gene knockout was confirmed. The primer set used is shown in Table 4. Total RNA was prepared using RNeasy Plus Mini Kit (QIAGEN). Digestion of gDNA was performed at 37° C. for 5 minutes by mixing 1 μL of 10× ezDNase buffer, 1 μL of ezDNase enzyme, 1 μL of template RNA (500 ng/μL), and 7 μL of water. In addition, for RT-PCR, 10 μL of template RNA (digested gDNA), 25 μL of 2× Platinum SuperFi RT-PCR Master Mix, 2.5 μL of Primer Set I Mixture (each 10 μM), 2.5 μL of Primer Set V Mixture (each 10 μM), 0.5 μL of SuperScript IV RT Mix, and 9.5 μL of water were mixed. The reaction was carried out at 60° C. for 10 minutes, 98° C. for 2 minutes, further 40 cycles of 98° C. for 10 seconds, 62° C. for 10 seconds, and 72° C. for 1 minute, followed by 72° C. for 5 minutes.

TABLE 4

| | | | SEQ |
|---|---|---|---|
| PCR | Primer | Sequence | ID NO |
| Primer set I | (RT)Human beta Actin-F | CACCAACTGGGACGACAT | 141 |
| | (RT)Human beta Actin-R | ACAGCCTGGATAGCAACG | 142 |
| Primer set V | (RT)Human FUS-F_3 | CTTATGGCCAGAGCCAGAACA | 143 |
| | (RT)Human FUS-R 3 | ATCATGGGCTGTCCCGTTTT | 144 |

### Example 5

Production of Co-Expression HEK293 Cell Strains of TurboGFP-Fused FUS_{wild-type} and TurboFP635-fused FUS_{P525L}

TurboGFP-fused FUS_{wild-type} and TurboFP635-fused FUS_{P525L} were cloned into a multi-cloning site of pAAVS1-puro-DNR (Origene). PAAVS1-puro-DNR (Origene)_TurboGFP-FUS_{wild-type}, pAAVS1-puro-DNR (Origene)_TurboFP635-FUS_{P525L}, and pCas-Guide-AAVS1 (Origene) were transfected into previously prepared FUS KO HEK293 cells to produce cells that co-express TurboGFP-fused FUS_{wild-type} and TurboFP635-fused FUS_{P525L}. Cloning of the cell strains was carried out by sorting double positive cells of TurboGFP and TurboFP635 with On-chip Sort (On-chip Biotechnologies Co., Ltd.), then fractionating single cells into 384 plates with On-chip SPiS (On-chip Biotechnology Co. Ltd.), and culturing.

### Example 6

Evaluation of RNA Interference Using Co-Expression HEK293 Cell Strains of TurboGFP-Fused FUS_{wild-type} and TurboFP635-Fused FUS_{P525L} (Imaging)

25 μL of a mixed solution of 25 μL of Opti-MEM (Invitrogen) and 1.5 μL of Lipofectamine© RNAi MAX (Invitrogen), and 25 μL of a mixed solution of 25 μL of Opti-MEM (Invitrogen) and 0.5 μL of 10 μM siRNA were mixed and incubated at room temperature for 15 to 20 minutes. The co-expression HEK293 Cell Strains of TurboGFP-fused FUS_{wild-type} and TurboFP635-fused FUS_{P525L} were suspended at 3.0×10⁵ cells/mL in a medium (FluoroBrite® DMEM containing 5% FBS, the same applies below.) warmed to 37° C. 100 μL of the cell suspension was mixed with 10 μL of Lipofectamine-siRNA complex prepared in advance, and the mixture was seeded on a 96-well plate (CellCarrier Ultra, collagen-coated, PerkinElmer), and cultured under the condition of 37° C. and 5% CO₂ (the following culture was performed under the same conditions). 100 μL of medium was added 24 hours after transfection, and data were acquired using an Operetta CLS© high-content confocal imaging system (PerkinElmer, lens immersed in 20× water, confocal mode) 48 hours after transfection. The total number of cells (number of nuclei), the number of TurboGFP positive cells, and the number of TurboFP635 positive cells were counted from the obtained image data, and the rate of TurboGFP positive cells (the number of TurboGFP positive cells/the total number of cells) and the rate of TurboFP635 positive cells (the number of TurboFP635 positive cells/the total number of cells) were calculated.

A TurboGFP positive cells and a TurboFP635 positive cells are defined as follows:

A TurboGFP positive cell (FUS_{wild-type} expressing cells): The cell with a value of 400 or more of the total fluorescence intensity of TurboGFP in nuclear region divided by the area (pixels) of the nuclear region.

A TurboFP635 positive cell (FUS_{P525L} expressing cells): The cell with a value of 400 or more of the total fluorescence intensity of TurboFP634 in cytoplasmic region divided by the area (pixels) of the cytoplasmic region.

Each rate of positive cells was substituted into the following formula to calculate the relative values of expression rate and silencing rate:

$$Expression rate (%) = 100 \times \{ ( rate of TurboGFP positive cells after treatment with various siRNA ) - ( rate of TurboGFP positive cells after treatment with positive control siRNA ) \} / \{ ( rate of TurboGFP positive cells after treatment with negative control siRNA ) - ( rate of TurboGFP positive cells after treatment with positive control siRNA ) \} , and$$

$$Silencing rate (%) = 100 \times \{ ( rate of TurboGFP positive cells after treatment with various siRNA ) - ( rate of TurboGFP positive cells after treatment with negative control siRNA ) \} / \{ ( rate of TurboGFP positive cells after treatment with positive control siRNA ) - ( rate of TurboGFP positive cells after treatment with negative control siRNA ) \} .$$

The above formula is for the case of FUS_{wild-type}. However, for the case of FUS_{P525L}, the same calculation can be performed from the rate of TurboFP635 positive cells. In the above formula, the negative control siRNA is an siRNA with a sequence that is not similar to a known gene sequence of humans, mice, and rats, and is provided by Horizon Discovery. The sequence is UAGCGACUAAACACAUCAA (SEQ ID NO: 145). On the other hand, the positive control siRNA is a mixture of four types of siRNA (SEQ ID NO: 146 to 149) designed to target arbitrary regions of human FUS mRNA, and is provided by Horizon Discovery. The sequences of the four types are as follows:

(SEQ ID NO: 146)
CCUACGGACAGCAGAGUUA
(SEQ ID NO: 147)
GAUUAUACCCAACAAGCAA
(SEQ ID NO: 148)
GAUCAAUCCUCCAUGAGUA
(SEQ ID NO: 149)
CGGGACAGCCCAUGAUUAA

The effects of the chemically modified siRNAs listed in Table 1 on the expression rates of wild-type FUS and FUS P525L mutation, as well as their silencing effects on expression and respective selectivity are shown in Tables 5 to 9. Tables 5 to 9 show the results of experiments carried out independently of each other, the results for the same siRNAs may be shown in each table, accordingly.

The same siRNAs are siRNA-010 and siRNA-010-4, etc.

Here, the selectivity is defined as follows:

$$selectivity in expression rate = expression rate of wild - type FUS ( \% ) / expression rate of FUS P 525 L mutation ( \% )$$

$$Selectivity in silencing rate = silencing rate of FUS P 525 L mutation ( \% ) - silencing rate of wild - type FUS ( \% )$$

TABLE 5

| | Expression rate (%) | | | Silencing rate (%) | | |
|---|---|---|---|---|---|---|
| | Wild- | FUS | | Wild- | FUS | |
| | type | P525L | Selec- | type | P525L | Selec- |
| siRNA | FUS | mutation | tivity | FUS | mutation | tivity |
| siRNA-010 | 38.1 | 6.3 | 6.1 | 61.9 | 93.7 | 31.8 |
| siRNA-010-2 | 27.6 | 18.1 | 1.5 | 72.4 | 81.9 | 9.5 |
| siRNA-010-4 | 50.7 | 33.2 | 1.5 | 49.3 | 66.8 | 17.6 |
| siRNA-010-5 | 54.7 | 36.5 | 1.5 | 45.3 | 63.5 | 18.2 |
| siRNA-010-12 | 25.4 | 16.4 | 1.5 | 74.6 | 83.6 | 9.0 |

TABLE 6

| | Expression rate (%) | | | Silencing rate (%) | | |
|---|---|---|---|---|---|---|
| | | FUS | | | FUS | |
| | Wild- | P525L | | Wild- | P525L | |
| | type | muta- | Selec- | type | muta- | Selec- |
| siRNA | FUS | tion | tivity | FUS | tion | tivity |
| siRNA-010 | 44.3 | 12.0 | 3.7 | 55.7 | 88.0 | 32.3 |
| siRNA-010-kh | 95.3 | 65.3 | 1.5 | 4.7 | 34.7 | 30.0 |
| siRNA-010-4 | 52.8 | 18.6 | 2.8 | 47.2 | 81.4 | 34.1 |
| siRNA-010-4-3 | 25.8 | 17.3 | 1.5 | 74.2 | 82.7 | 8.5 |
| siRNA-010-4-4 | 82.9 | 62.4 | 1.3 | 17.1 | 37.6 | 20.5 |
| siRNA-010-4-7 | 71.2 | 42.7 | 1.7 | 28.8 | 57.3 | 28.5 |
| siRNA-010-16 | 92.9 | 57.0 | 1.6 | 7.1 | 43.0 | 35.9 |
| siRNA-010-16-2 | 89.2 | 66.2 | 1.3 | 10.8 | 33.8 | 23.0 |
| siRNA-010-16-7 | 26.5 | 17.5 | 1.5 | 73.5 | 82.5 | 9.0 |
| siRNA-010-16-9 | 76.9 | 55.5 | 1.4 | 23.1 | 44.5 | 21.4 |
| siRNA-010-16-11 | 30.9 | 19.6 | 1.6 | 69.1 | 80.4 | 11.3 |
| siRNA-010-16-12 | 27.0 | 15.5 | 1.7 | 73.0 | 84.5 | 11.5 |
| siRNA-010-4-14 | 68.8 | 45.5 | 1.5 | 31.2 | 54.5 | 23.3 |
| siRNA-010-4-15 | 86.0 | 61.9 | 1.4 | 14.0 | 38.1 | 24.1 |
| siRNA-010-4-16 | 84.2 | 57.8 | 1.5 | 15.8 | 42.2 | 26.5 |
| siRNA-010-4-18 | 87.2 | 55.7 | 1.6 | 12.8 | 44.3 | 31.5 |
| siRNA-010-4-19 | 91.9 | 64.0 | 1.4 | 8.1 | 36.0 | 27.9 |

TABLE 7

| | Expression rate (%) | | | silencing rate (%) | | |
|---|---|---|---|---|---|---|
| | | FUS | | | FUS | |
| | Wild- | P525L | | Wild- | P525L | |
| | type | muta- | Selec- | type | muta- | Selec- |
| siRNA | FUS | tion | tivity | FUS | tion | tivity |
| siRNA-002 | 59.6 | 41.2 | 1.4 | 40.4 | 58.8 | 18.5 |
| siRNA-002-4 | 87.8 | 63.0 | 1.4 | 12.2 | 37.0 | 24.9 |
| siRNA-002-12 | 86.4 | 63.5 | 1.4 | 13.6 | 36.5 | 22.9 |
| siRNA-002-16 | 88.7 | 60.0 | 1.5 | 11.3 | 40.0 | 28.7 |
| siRNA-003 | 92.5 | 58.5 | 1.6 | 7.5 | 41.5 | 34.0 |
| siRNA-003-4 | 100.5 | 68.9 | 1.5 | −0.5 | 31.1 | 31.6 |
| siRNA-003-16 | 79.4 | 59.3 | 1.3 | 20.6 | 40.7 | 20.1 |
| siRNA-006 | 51.8 | 22.5 | 2.3 | 48.2 | 77.5 | 29.3 |
| siRNA-006-4 | 91.3 | 59.3 | 1.5 | 8.7 | 40.7 | 32.0 |
| siRNA-006-8 | 87.5 | 64.2 | 1.4 | 12.5 | 35.8 | 23.3 |
| siRNA-006-12 | 89.1 | 65.9 | 1.4 | 10.9 | 34.1 | 23.2 |
| siRNA-008 | 58.7 | 28.7 | 2.0 | 41.3 | 71.3 | 30.0 |
| siRNA-008-16 | 73.0 | 46.5 | 1.6 | 27.0 | 53.5 | 26.5 |
| siRNA-009 | 61.3 | 29.7 | 2.1 | 38.7 | 70.3 | 31.6 |
| siRNA-009-4 | 89.2 | 58.0 | 1.5 | 10.8 | 42.0 | 31.2 |
| siRNA-009-16 | 77.3 | 56.9 | 1.4 | 22.7 | 43.1 | 20.3 |
| siRNA-011 | 35.9 | 16.7 | 2.2 | 64.1 | 83.3 | 19.2 |
| siRNA-011-4 | 67.9 | 46.9 | 1.4 | 32.1 | 53.1 | 21.0 |
| siRNA-011-8 | 79.1 | 52.3 | 1.5 | 20.9 | 47.7 | 26.8 |
| siRNA-011-12 | 77.2 | 46.9 | 1.6 | 22.8 | 53.1 | 30.2 |

TABLE 8

| | Expression rate (%) | | | Silencing rate (%) | | |
|---|---|---|---|---|---|---|
| | | FUS | | | FUS | |
| | Wild- | P525L | | Wild- | P525L | |
| | type | muta- | Selec- | type | muta- | Selec- |
| siRNA | FUS | tion | tivity | FUS | tion | tivity |
| siRNA-010 | 48.6 | 4.1 | 11.7 | 51.4 | 95.9 | 44.5 |
| siRNA-010-4 | 50.0 | 27.4 | 1.8 | 50.0 | 72.6 | 22.5 |
| siRNA-010-8 | 23.4 | 6.8 | 3.4 | 76.6 | 93.2 | 16.6 |
| siRNA-010-4-4 | 82.4 | 56.5 | 1.5 | 17.6 | 43.5 | 25.9 |
| siRNA-010-4-13 | 28.7 | 7.7 | 3.7 | 71.3 | 92.3 | 21.0 |
| siRNA-010-4-8 | 102.5 | 59.5 | 1.7 | −2.5 | 40.5 | 42.9 |
| siRNA-010-4-23 | 86.7 | 66.6 | 1.3 | 13.3 | 33.4 | 20.0 |
| siRNA-010-4-24 | 95.8 | 66.9 | 1.4 | 4.2 | 33.1 | 29.0 |
| siRNA-010-16 | 110.0 | 40.6 | 2.7 | −10.0 | 59.4 | 69.5 |
| siRNA-010-16-6 | 69.7 | 20.2 | 3.4 | 30.3 | 79.8 | 49.5 |
| siRNA-010-16-12 | 53.4 | 11.6 | 4.6 | 46.6 | 88.4 | 41.8 |
| siRNA-010-16-9 | 124.7 | 58.9 | 2.1 | −24.7 | 41.1 | 65.7 |
| siRNA-010-16-13 | 44.1 | 16.0 | 2.8 | 55.9 | 84.0 | 28.1 |
| siRNA-010-16-14 | 85.1 | 21.8 | 3.9 | 14.9 | 78.2 | 63.3 |
| siRNA-010-16-15 | 53.4 | 18.9 | 2.8 | 46.6 | 81.1 | 34.5 |
| siRNA-010-16-16 | 98.1 | 21.6 | 4.5 | 1.9 | 78.4 | 76.5 |

TABLE 9

| | Expression rate (%) | | | Silencing rate (%) | | |
|---|---|---|---|---|---|---|
| | | FUS | | | FUS | |
| | Wild- | P525L | | Wild- | P525L | |
| | type | muta- | Selec- | type | muta- | Selec- |
| siRNA | FUS | tion | tivity | FUS | tion | tivity |
| siRNA-003 | 104.1 | 58.7 | 1.8 | −4.1 | 41.3 | 45.4 |
| siRNA-003-16-13 | 111.4 | 69.4 | 1.6 | −11.4 | 30.6 | 42.1 |
| siRNA-006 | 63.0 | 25.6 | 2.5 | 37.0 | 74.4 | 37.4 |
| siRNA-006-16 | 84.5 | 63.9 | 1.3 | 15.5 | 36.1 | 20.6 |
| siRNA-006-16-13 | 70.3 | 29.5 | 2.4 | 29.7 | 70.5 | 40.8 |
| siRNA-006-16-14 | 70.6 | 35.0 | 2.0 | 29.4 | 65.0 | 35.5 |
| siRNA-006-16-15 | 62.7 | 35.4 | 1.8 | 37.3 | 64.6 | 27.4 |
| siRNA-006-16-16 | 70.4 | 37.1 | 1.9 | 29.6 | 62.9 | 33.3 |
| siRNA-008 | 52.8 | 30.5 | 1.7 | 47.2 | 69.5 | 22.3 |
| siRNA-008-16-16 | 94.5 | 68.3 | 1.4 | 5.5 | 31.7 | 26.2 |
| siRNA-009 | 47.1 | 17.2 | 2.7 | 52.9 | 82.8 | 29.9 |
| siRNA-009-16-13 | 64.0 | 20.3 | 3.2 | 36.0 | 79.7 | 43.8 |
| siRNA-009-16-14 | 75.0 | 23.5 | 3.2 | 25.0 | 76.5 | 51.5 |
| siRNA-009-16-15 | 68.8 | 23.5 | 2.9 | 31.2 | 76.5 | 45.2 |
| siRNA-009-16-16 | 76.9 | 37.3 | 2.1 | 23.1 | 62.7 | 39.6 |
| siRNA-011 | 45.9 | 15.5 | 3.0 | 54.1 | 84.5 | 30.4 |
| siRNA-011-4 | 87.2 | 62.5 | 1.4 | 12.8 | 37.5 | 24.7 |
| siRNA-011-16-13 | 98.5 | 44.0 | 2.2 | 1.5 | 56.0 | 54.5 |
| siRNA-011-16-14 | 102.4 | 46.1 | 2.2 | −2.4 | 53.9 | 56.3 |
| siRNA-011-16-15 | 100.0 | 46.3 | 2.2 | 0.0 | 53.7 | 53.6 |
| siRNA-011-16-16 | 108.7 | 42.7 | 2.5 | −8.7 | 57.3 | 66.0 |

As shown in Tables 5 to 9, each of the chemically modified siRNA has selectivity in FUS expression rate of 1.5-fold or more and/or selectivity in FUS silencing rate of 20% or more, and silencing rate of FUS P525L mutation of 30% or more. Therefore, these chemically modified siRNAs are suggested to have equivalent silencing effect on expression and high selectivity to FUS P525L mutation compared to naked siRNA.

As shown in Tables 6, 8 and 9, each of siRNA-010-16, siRNA-010-16-12, siRNA-010-8, siRNA-010-4-13, siRNA-010-16-6, siRNA-010-16-13, siRNA-010-16-14, siRNA-010-16-15, siRNA-010-16-16, siRNA-006-16-13, siRNA-006-16-14, siRNA-006-16-15, siRNA-006-16-16, siRNA-009-16-15, siRNA-009-16-16, siRiNA-011-16-13, siRNA-011-16-14, siRNA-011-16-15, and siRNA-011-16-16 has selectivity in FUS expression rate of 2-fold or more and/or selectivity in FUS silencing rate of 40% or more, and silencing rate of FUS P525L mutation of 50% or more. Therefore, these chemically modified siRNAs are suggested to have equivalent silencing effect on expression and high selectivity to FUS P525L mutation compared to naked siRNA.

### Example 7

Evaluation of RNA Interference Using Co-Expression HEK293 Cell Strains of TurboGFP-Fused FUS_{wild-type} and TurboFP635-Fused FUS_{P525L} (Real-Time PCR)

siRNA was transfected into the cells according to the method shown in Example 6. 48 hours after the transfected, the medium was removed completely, and 50 μL of cell lysate prepared by mixing 0.5 μL of DNase I (Life Technologies Japan) and 49.5 μL of Lysis Solution (Life Technologies Japan) was added and incubated at room temperature for 5 minutes. After that, 5 μL of Stop Solution (Life Technologies Japan) was added and mixed, and then incubated at room temperature for 2 minutes, and this was subjected to reverse transcription reaction. To a reverse transcription reaction solution consisting of 25 μL of 2× Fast Advanced RT Buffer (Life Technologies Japan), 2.5 μL of 20× Fast Advanced RT Enzyme Mix (Life Technologies Japan), and 12.5 μL of Nuclease-free Water, 10 μL of a cell lysate prepared in advance was added, and reacted at 37° C. for 30 minutes and then at 95° C. for 5 minutes to synthesize cDNA.

As for real-time PCR, three genes, TurboGFP-fused FUS_{wild-type}, TurboFP635-fused FUS_{P525L} and endogenous control gene (using GAPDH) were detected in the same reaction system. A reaction solution was prepared by mixing 10 μL of TaqMan® Fast Advanced Master Mix (Life Technologies Japan), 0.06 μL each of 100 μM primers (GFP_X_F, GFP_X_R, FP635_X_F, FP635_X_R), 0.5 μL each of 10 μM TaqMan probes (TurboGFP (NED) and TurboFP635 (FAM)), 1.0 μL of 20× TaqMan Assay (GAPDH) (Life Technologies Japan), 3.76 L of nuclease-free water, and 4 μL of cDNA prepared in advance. This reaction solution was reacted in a real-time PCR device (QuantStudio 7 pro, Life Technology Japan) at 50° C. for 2 minutes, then at 95° C. for 20 seconds, followed by 40 cycles of 95° C. for 1 second and 60° C. for 20 seconds. The primers and TaqMan probes used in the real-time PCR are shown in Tables 10 and 11, respectively. Gene expression levels were calculated as relative values according to ΔΔCt method. Here, the relative mRNA expression rate was calculated by setting the mRNA expression rate after treatment with negative control siRNA as 100% and the mRNA expression rate after treatment with positive control siRNA as 0%.

TABLE 10

| Primer | Sequence | SEQ ID NO |
|---|---|---|
| GFP_X_F | TGGGCATCGTGGAGTACCA | 150 |
| GFP_X_R | TGCTTGTTGGGTATAATCGTTTGA | 151 |
| FP635_X_F | CTGCGACCTGCCTAGCAAAC | 152 |
| FP635_X_R | CCATAGCTTTGGGTTGCTTGTT | 153 |

TABLE 11

| Probe | Sequence | SEQ ID NO |
|---|---|---|
| TurboGFP (NED) | AGACCCCGGATGCAG | 154 |
| TurboFP635 (FAM) | ACAGCTCCGGACTCA | 155 |

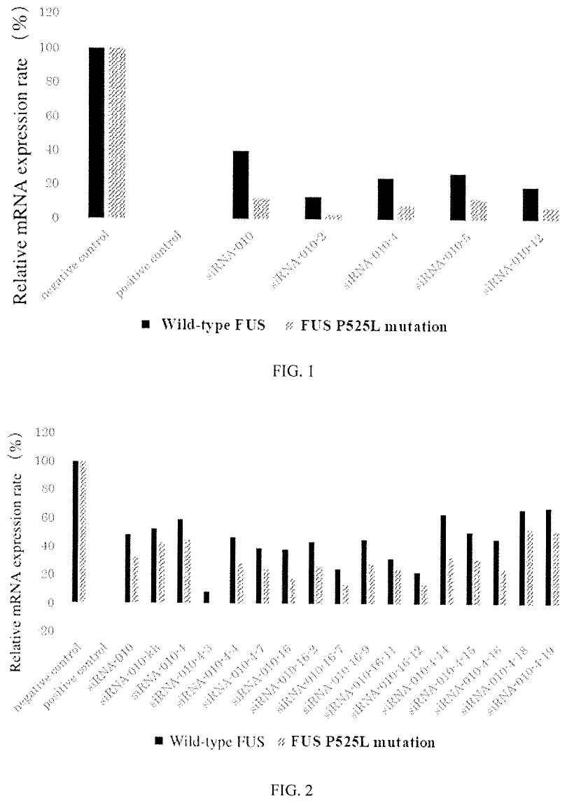

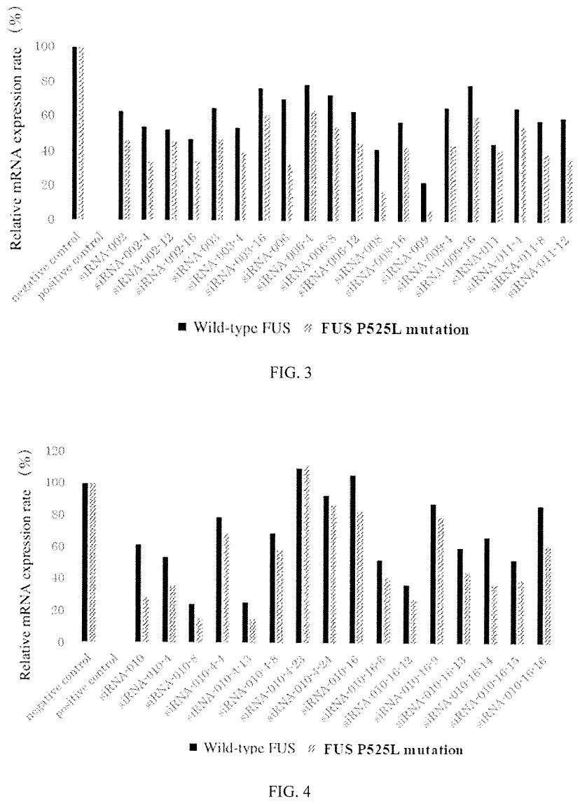

The effects of the chemically modified siRNA described in the specification of the present application on the mRNA expression rates of wild-type FUS and FUS P525L mutation are shown in FIGS. 1 to 5. Similarly to the results of quantitative analysis of the expression levels of FUS protein by the imaging analysis of Example 6, the results of quantitative analysis of the expression levels of mRNA by real-time PCR also suggest that each chemically modified siRNA maintains high selectivity for silencing of FUS P525L mutation.

### Example 8

### Evaluation of Stability in Human Serum

The stability of naked siRNA and the chemically modified siRNA described in the specification of the present application in human serum was evaluated. 10% (v/v) human serum was prepared by adding 100 μL of human serum (Cosmo Bio Co., Ltd.) to 900 μL of PBS (phosphate buffered saline) and mixing them. 5 μL of 100 μM siRNA was mixed with 95 μL of 10% (v/v) human serum warmed to 37° C. and incubated at 37° C. After the start of the incubation, 2 μL each of the samples was sampled at each designated time, mixed with 18 μL of 1×TBE Sample Buffer and cryopreserved immediately. The sampling times for each siRNA were 0, 15, 30, 45, 60, 75, and 90 minutes for naked siRNA (siRNA-006, siRNA-009, siRNA-010, and siRNA-011), and 0, 1, 3, 6, and 24 hours for the chemically modified siRNA. The frozen samples were thawed, and 5 μL each of the samples was electrophoresed (150CV, 40 minutes) in 20% TBE-PAGE and 1×TBE Buffer, gel-stained with the SYBER® Gold (Thermo Fisher Scientific), and detected with an Amersham Imager 680 UV transilluminator 312 nm (cytiva).

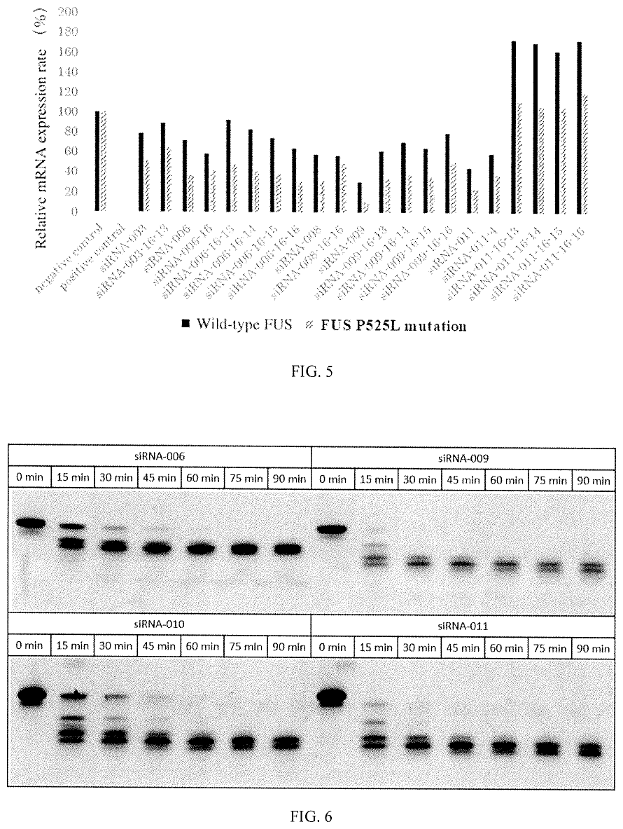

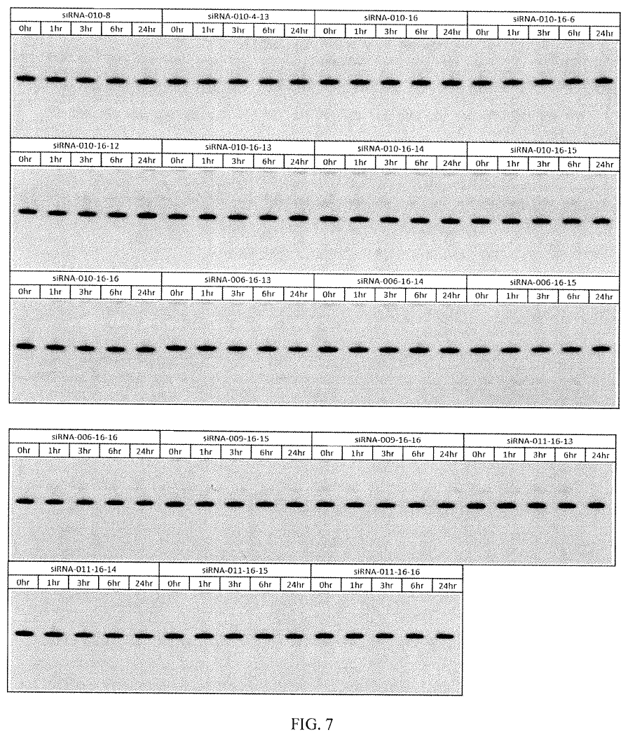

The results of the stability in human serum from the investigation are shown in FIG. 6 (for naked siRNA) and FIG. 7 (for the chemically modified siRNA). The stability in human serum was confirmed by the presence or absence of band shifts and multiple bands due to incubation, relative to the bands observed without incubation (0 min). As a result, a band shift and multiple bands were observed within 1 hour of the start of incubation for naked siRNA, indicating that degradation by RNase proceeded rapidly. On the other hand, no clear band shift was observed for the chemically modified siRNA even 24 hours after the start of the incubation, indicating that no degradation by RNase occurred. Therefore, it was demonstrated that the chemically modified siRNA of the present disclosure has dramatically improved stability against RNases.

1. A chemically modified siRNA or a salt thereof consisting of a sense strand and an antisense strand, wherein the antisense strand comprises a region complementary or substantially complementary to a part of mRNA encoding FUS P525L mutation protein, the complementary region is 19 to 21 nucleotides in length, the siRNA comprises at least one substitution selected from the group consisting of 2′-F-nucleotides, 2′-OMe-nucleotides, nucleotides in which a 2′-0 atom and a 4′-C atom are bridged with methylene, 2′-deoxy-nucleotides, and phosphorothioate bonds that form internucleotide linkage.

2. The chemically modified siRNA or a salt thereof according to claim 1, comprising a motif of two or four consecutive 2′-F-nucleotides or 2′-OMe-nucleotides at or adjacent to the site of cleavage of the RNA strand by Ago2, and comprising 2′-F-nucleotides and 2′-OMe-nucleotides alternately along the RNA strand except the motif.

3. The chemically modified siRNA or a salt thereof according to claim 2, wherein the siRNA is represented by formula (I) below:
(I)
Sense strand:
5′-(YX)a-(YY)b-(XY)c-(XX)d-(YX)e-(YY)f-(XY)g-
(XYX)h-(YXY)i-3′
Antisense strand:
3′-(YX)j-(XY)k-(XY)a-(XX)b-(YX)c-(YY)d-(XY)e-
(XX)f-(YX)g-(Y)h-(X)i-5′
(wherein, X and Y are 2′-F-nucleotides and 2′-OMe-nucleotides, respectively;
a, b, c, d, e, f, g, h, i, j, and k are each independently an integer of 0 to 4;
(a, b, c, j, k) are (0, 0, 4, 1, 0), (0, 1, 3, 0, 1), (0, 2, 2, 0, 1), (1, 1, 2, 0, 1), (2, 1, 1, 0, 1), (1, 2, 1, 0, 1), (2, 2, 0, 0, 1), or (3, 1, 0, 0, 1) in this order;
(e, f, g, h, i) are (4, 0, 0, 0, 1), (3, 1, 0, 1, 0), (2, 2, 0, 1, 0), (2, 1, 1, 1, 0), (1, 1, 2, 1, 0), (1, 2, 1, 1, 0), (0, 2, 2, 1, 0), or (0, 1, 3, 1, 0) in this order when d is 1;
(e, f, g, h, i) are (3, 0, 0, 0, 1), (2, 1, 0, 1, 0), (1, 2, 0, 1, 0), (1, 1, 1, 1, 0), (0, 2, 1, 1, 0), or (0, 1, 2, 1, 0) in this order when d is 2);
and is 21 nucleotides in length.

4. The chemically modified siRNA or a salt thereof according to claim 2, selected from the group consisting of the double-stranded RNA consisting of the sense strand of SEQ ID NO: 5 and the antisense strand of SEQ ID NO: 6, the double-stranded RNA consisting of the sense strand of SEQ ID NO: 13 and the antisense strand of SEQ ID NO: 14, the double-stranded RNA consisting of the sense strand of SEQ ID NO: 15 and the antisense strand of SEQ ID NO: 16, the double-stranded RNA consisting of the sense strand of SEQ ID NO: 17 and the antisense strand of SEQ ID NO: 18, the double-stranded RNA consisting of the sense strand of SEQ ID NO: 19 and the antisense strand of SEQ ID NO: 20, the double-stranded RNA consisting of the sense strand of SEQ ID NO: 21 and the antisense strand of SEQ ID NO: 22, the double-stranded RNA consisting of the sense strand of SEQ ID NO: 23 and the antisense strand of SEQ ID NO: 24, the double-stranded RNA consisting of the sense strand of SEQ ID NO: 35 and the antisense strand of SEQ ID NO: 36, the double-stranded RNA consisting of the sense strand of SEQ ID NO: 37 and the antisense strand of SEQ ID NO: 38, the double-stranded RNA consisting of the sense strand of SEQ ID NO: 39 and the antisense strand of SEQ ID NO: 40, the double-stranded RNA consisting of the sense strand of SEQ ID NO: 41 and the antisense strand of SEQ ID NO: 42, the double-stranded RNA consisting of the sense strand of SEQ ID NO: 43 and the antisense strand of SEQ ID NO: 44, the double-stranded RNA consisting of the sense strand of SEQ ID NO: 45 and the antisense strand of SEQ ID NO: 46, the double-stranded RNA consisting of the sense strand of SEQ ID NO: 47 and the antisense strand of SEQ ID NO: 48, the double-stranded RNA consisting of the sense strand of SEQ ID NO: 49 and the antisense strand of SEQ ID NO: 50, the double-stranded RNA consisting of the sense strand of SEQ ID NO: 51 and the antisense strand of SEQ ID NO: 52, the double-stranded RNA consisting of the sense strand of SEQ ID NO: 53 and the antisense strand of SEQ ID NO: 54, the double-stranded RNA consisting of the sense strand of SEQ ID NO: 55 and the antisense strand of SEQ ID NO: 56, the double-stranded RNA consisting of the sense strand of SEQ ID NO: 57 and the antisense strand of SEQ ID NO: 58, the double-stranded RNA consisting of the sense strand of SEQ ID NO: 59 and the antisense strand of SEQ ID NO: 60, the double-stranded RNA consisting of the sense strand of SEQ ID NO: 63 and the antisense strand of SEQ ID NO: 64, the double-stranded RNA consisting of the sense strand of SEQ ID NO: 67 and the antisense strand of SEQ ID NO: 68, the double-stranded RNA consisting of the sense strand of SEQ ID NO: 71 and the antisense strand of SEQ ID NO: 72, the double-stranded RNA consisting of the sense strand of SEQ ID NO: 73 and the antisense strand of SEQ ID NO: 74, the double-stranded RNA consisting of the sense strand of SEQ ID NO: 75 and the antisense strand of SEQ ID NO: 76, the double-stranded RNA consisting of the sense strand of SEQ ID NO: 79 and the antisense strand of SEQ ID NO: 80, the double-stranded RNA consisting of the sense strand of SEQ ID NO: 81 and the antisense strand of SEQ ID NO: 82, the double-stranded RNA consisting of the sense strand of SEQ ID NO: 85 and the antisense strand of SEQ ID NO: 86, the double-stranded RNA consisting of the sense strand of SEQ ID NO: 87 and the antisense strand of SEQ ID NO: 88, the double-stranded RNA consisting of the sense strand of SEQ ID NO: 89 and the antisense strand of SEQ ID NO: 90, the double-stranded RNA consisting of the sense strand of SEQ ID NO: 91 and the antisense strand of SEQ ID NO: 92, the double-stranded RNA consisting of the sense strand of SEQ ID NO: 93 and the antisense strand of SEQ ID NO: 94, the double-stranded RNA consisting of the sense strand of SEQ ID NO: 97 and the antisense strand of SEQ ID NO: 98, the double-stranded RNA consisting of the sense strand of SEQ ID NO: 99 and the antisense strand of SEQ ID NO: 100, the double-stranded RNA consisting of the sense strand of SEQ ID NO: 103 and the antisense strand of SEQ ID NO: 104, the double-stranded RNA consisting of the sense strand of SEQ ID NO: 105 and the antisense strand of SEQ ID NO: 106, the double-stranded RNA consisting of the sense strand of SEQ ID NO: 107 and the antisense strand of SEQ ID NO: 108, the double-stranded RNA consisting of the sense strand of SEQ ID NO: 109 and the antisense strand of SEQ ID NO: 110, the double-stranded RNA consisting of the sense strand of SEQ ID NO: 111 and the antisense strand of SEQ ID NO: 112, the double-stranded RNA consisting of the sense strand of SEQ ID NO: 113 and the antisense strand of SEQ ID NO: 114, the double-stranded RNA consisting of the sense strand of SEQ ID NO: 117 and the antisense strand of SEQ ID NO: 118, the double-stranded RNA consisting of the sense strand of SEQ ID NO: 119 and the antisense strand of SEQ ID NO: 120, the double-stranded RNA consisting of the sense strand of SEQ ID NO: 123 and the antisense strand of SEQ ID NO: 124, the double-stranded RNA consisting of the sense strand of SEQ ID NO: 125 and the antisense strand of SEQ ID NO: 126, the double-stranded RNA consisting of the sense strand of SEQ ID NO: 127 and the antisense strand of SEQ ID NO: 128, and the double-stranded RNA consisting of the sense strand of SEQ ID NO: 129 and the antisense strand of SEQ ID NO: 130.

5. The chemically modified siRNA or a salt thereof according to claim 2, selected from the group consisting of the double-stranded RNA consisting of the sense strand of SEQ ID NO: 13 and the antisense strand of SEQ ID NO: 14, the double-stranded RNA consisting of the sense strand of SEQ ID NO: 21 and the antisense strand of SEQ ID NO: 22, the double-stranded RNA consisting of the sense strand of SEQ ID NO: 39 and the antisense strand of SEQ ID NO: 40, the double-stranded RNA consisting of the sense strand of SEQ ID NO: 43 and the antisense strand of SEQ ID NO: 44, the double-stranded RNA consisting of the sense strand of SEQ ID NO: 49 and the antisense strand of SEQ ID NO: 50, the double-stranded RNA consisting of the sense strand of SEQ ID NO: 53 and the antisense strand of SEQ ID NO: 54, the double-stranded RNA consisting of the sense strand of SEQ ID NO: 55 and the antisense strand of SEQ ID NO: 56, the double-stranded RNA consisting of the sense strand of SEQ ID NO: 57 and the antisense strand of SEQ ID NO: 58, the double-stranded RNA consisting of the sense strand of SEQ ID NO: 59 and the antisense strand of SEQ ID NO: 60, the double-stranded RNA consisting of the sense strand of SEQ ID NO: 87 and the antisense strand of SEQ ID NO: 88, the double-stranded RNA consisting of the sense strand of SEQ ID NO: 89 and the antisense strand of SEQ ID NO: 90, the double-stranded RNA consisting of the sense strand of SEQ ID NO: 91 and the antisense strand of SEQ ID NO: 92, the double-stranded RNA consisting of the sense strand of SEQ ID NO: 93 and the antisense strand of SEQ ID NO: 94, the double-stranded RNA consisting of the sense strand of SEQ ID NO: 111 and the antisense strand of SEQ ID NO: 112, the double-stranded RNA consisting of the sense strand of SEQ ID NO: 113 and the antisense strand of SEQ ID NO: 114, the double-stranded RNA consisting of the sense strand of SEQ ID NO: 123 and the antisense strand of SEQ ID NO: 124, the double-stranded RNA consisting of the sense strand of SEQ ID NO: 125 and the antisense strand of SEQ ID NO: 126, the double-stranded RNA consisting of the sense strand of SEQ ID NO: 127 and the antisense strand of SEQ ID NO: 128, and the double-stranded RNA consisting of the sense strand of SEQ ID NO: 129 and the antisense strand of SEQ ID NO: 130.

6. The chemically modified siRNA or a salt thereof according to claim 1, selected from the group consisting of the double-stranded RNA consisting of the sense strand of SEQ ID NO: 3 and the antisense strand of SEQ ID NO: 4, the double-stranded RNA consisting of the sense strand of SEQ ID NO: 7 and the antisense strand of SEQ ID NO: 8, the double-stranded RNA consisting of the sense strand of SEQ ID NO: 9 and the antisense strand of SEQ ID NO: 10, the double-stranded RNA consisting of the sense strand of SEQ ID NO: 11 and the antisense strand of SEQ ID NO: 12, the double-stranded RNA consisting of the sense strand of SEQ ID NO: 25 and the antisense strand of SEQ ID NO: 26, the double-stranded RNA consisting of the sense strand of SEQ ID NO: 27 and the antisense strand of SEQ ID NO: 28, the double-stranded RNA consisting of the sense strand of SEQ ID NO: 29 and the antisense strand of SEQ ID NO: 30, the double-stranded RNA consisting of the sense strand of SEQ ID NO: 31 and the antisense strand of SEQ ID NO: 32, the double-stranded RNA consisting of the sense strand of SEQ ID NO: 33 and the antisense strand of SEQ ID NO: 34, the double-stranded RNA consisting of the sense strand of SEQ ID NO: 65 and the antisense strand of SEQ ID NO: 66, the double-stranded RNA consisting of the sense strand of SEQ ID NO: 83 and the antisense strand of SEQ ID NO: 84, the double-stranded RNA consisting of the sense strand of SEQ ID NO: 121 and the antisense strand of SEQ ID NO: 122.

7. The chemically modified siRNA or a salt thereof according to claim 1, where in the three nucleotides located at 5′-end and 3′-end of sense strands and antisense strands, the adjacent internucleotide linkage are phosphorothioate bonds.

8. The chemically modified siRNA or a salt thereof according to claim 1 for silencing of FUS P525L mutation.

9. The chemically modified siRNA or a salt thereof according to claim 1 for selectively silencing of FUS P525L mutation without substantially silencing of wild-type FUS.

10. An inhibitor against the expression of FUS P525L mutation, containing the chemically modified siRNA or a salt thereof according to claim 1.

11. A pharmaceutical composition for prevention or treatment of ALS, containing the chemically modified siRNA or a salt thereof according to claim 1 as an active ingredient.

12. The pharmaceutical composition according to claim 11, wherein the ALS is ALS having FUS P525L mutation.

13. The chemically modified siRNA or a salt thereof according to claim 3, selected from the group consisting of the double-stranded RNA consisting of the sense strand of SEQ ID NO: 5 and the antisense strand of SEQ ID NO: 6, the double-stranded RNA consisting of the sense strand of SEQ ID NO: 13 and the antisense strand of SEQ ID NO: 14, the double-stranded RNA consisting of the sense strand of SEQ ID NO: 15 and the antisense strand of SEQ ID NO: 16, the double-stranded RNA consisting of the sense strand of SEQ ID NO: 17 and the antisense strand of SEQ ID NO: 18, the double-stranded RNA consisting of the sense strand of SEQ ID NO: 19 and the antisense strand of SEQ ID NO: 20, the double-stranded RNA consisting of the sense strand of SEQ ID NO: 21 and the antisense strand of SEQ ID NO: 22, the double-stranded RNA consisting of the sense strand of SEQ ID NO: 23 and the antisense strand of SEQ ID NO: 24, the double-stranded RNA consisting of the sense strand of SEQ ID NO: 35 and the antisense strand of SEQ ID NO: 36, the double-stranded RNA consisting of the sense strand of SEQ ID NO: 37 and the antisense strand of SEQ ID NO: 38, the double-stranded RNA consisting of the sense strand of SEQ ID NO: 39 and the antisense strand of SEQ ID NO: 40, the double-stranded RNA consisting of the sense strand of SEQ ID NO: 41 and the antisense strand of SEQ ID NO: 42, the double-stranded RNA consisting of the sense strand of SEQ ID NO: 43 and the antisense strand of SEQ ID NO: 44, the double-stranded RNA consisting of the sense strand of SEQ ID NO: 45 and the antisense strand of SEQ ID NO: 46, the double-stranded RNA consisting of the sense strand of SEQ ID NO: 47 and the antisense strand of SEQ ID NO: 48, the double-stranded RNA consisting of the sense strand of SEQ ID NO: 49 and the antisense strand of SEQ ID NO: 50, the double-stranded RNA consisting of the sense strand of SEQ ID NO: 51 and the antisense strand of SEQ ID NO: 52, the double-stranded RNA consisting of the sense strand of SEQ ID NO: 53 and the antisense strand of SEQ ID NO: 54, the double-stranded RNA consisting of the sense strand of SEQ ID NO: 55 and the antisense strand of SEQ ID NO: 56, the double-stranded RNA consisting of the sense strand of SEQ ID NO: 57 and the antisense strand of SEQ ID NO: 58, the double-stranded RNA consisting of the sense strand of SEQ ID NO: 59 and the antisense strand of SEQ ID NO: 60, the double-stranded RNA consisting of the sense strand of SEQ ID NO: 63 and the antisense strand of SEQ ID NO: 64, the double-stranded RNA consisting of the sense strand of SEQ ID NO: 67 and the antisense strand of SEQ ID NO: 68, the double-stranded RNA consisting of the sense strand of SEQ ID NO: 71 and the antisense strand of SEQ ID NO: 72, the double-stranded RNA consisting of the sense strand of SEQ ID NO: 73 and the antisense strand of SEQ ID NO: 74, the double-stranded RNA consisting of the sense strand of SEQ ID NO: 75 and the antisense strand of SEQ ID NO: 76, the double-stranded RNA consisting of the sense strand of SEQ ID NO: 79 and the antisense strand of SEQ ID NO: 80, the double-stranded RNA consisting of the sense strand of SEQ ID NO: 81 and the antisense strand of SEQ ID NO: 82, the double-stranded RNA consisting of the sense strand of SEQ ID NO: 85 and the antisense strand of SEQ ID NO: 86, the double-stranded RNA consisting of the sense strand of SEQ ID NO: 87 and the antisense strand of SEQ ID NO: 88, the double-stranded RNA consisting of the sense strand of SEQ ID NO: 89 and the antisense strand of SEQ ID NO: 90, the double-stranded RNA consisting of the sense strand of SEQ ID NO: 91 and the antisense strand of SEQ ID NO: 92, the double-stranded RNA consisting of the sense strand of SEQ ID NO: 93 and the antisense strand of SEQ ID NO: 94, the double-stranded RNA consisting of the sense strand of SEQ ID NO: 97 and the antisense strand of SEQ ID NO: 98, the double-stranded RNA consisting of the sense strand of SEQ ID NO: 99 and the antisense strand of SEQ ID NO: 100, the double-stranded RNA consisting of the sense strand of SEQ ID NO: 103 and the antisense strand of SEQ ID NO: 104, the double-stranded RNA consisting of the sense strand of SEQ ID NO: 105 and the antisense strand of SEQ ID NO: 106, the double-stranded RNA consisting of the sense strand of SEQ ID NO: 107 and the antisense strand of SEQ ID NO: 108, the double-stranded RNA consisting of the sense strand of SEQ ID NO: 109 and the antisense strand of SEQ ID NO: 110, the double-stranded RNA consisting of the sense strand of SEQ ID NO: 111 and the antisense strand of SEQ ID NO: 112, the double-stranded RNA consisting of the sense strand of SEQ ID NO: 113 and the antisense strand of SEQ ID NO: 114, the double-stranded RNA consisting of the sense strand of SEQ ID NO: 117 and the antisense strand of SEQ ID NO: 118, the double-stranded RNA consisting of the sense strand of SEQ ID NO: 119 and the antisense strand of SEQ ID NO: 120, the double-stranded RNA consisting of the sense strand of SEQ ID NO: 123 and the antisense strand of SEQ ID NO: 124, the double-stranded RNA consisting of the sense strand of SEQ ID NO: 125 and the antisense strand of SEQ ID NO: 126, the double-stranded RNA consisting of the sense strand of SEQ ID NO: 127 and the antisense strand of SEQ ID NO: 128, and the double-stranded RNA consisting of the sense strand of SEQ ID NO: 129 and the antisense strand of SEQ ID NO: 130.

14. The chemically modified siRNA or a salt thereof according to claim 3, selected from the group consisting of the double-stranded RNA consisting of the sense strand of SEQ ID NO: 13 and the antisense strand of SEQ ID NO: 14, the double-stranded RNA consisting of the sense strand of SEQ ID NO: 21 and the antisense strand of SEQ ID NO: 22, the double-stranded RNA consisting of the sense strand of SEQ ID NO: 39 and the antisense strand of SEQ ID NO: 40, the double-stranded RNA consisting of the sense strand of SEQ ID NO: 43 and the antisense strand of SEQ ID NO: 44, the double-stranded RNA consisting of the sense strand of SEQ ID NO: 49 and the antisense strand of SEQ ID NO: 50, the double-stranded RNA consisting of the sense strand of SEQ ID NO: 53 and the antisense strand of SEQ ID NO: 54, the double-stranded RNA consisting of the sense strand of SEQ ID NO: 55 and the antisense strand of SEQ ID NO: 56, the double-stranded RNA consisting of the sense strand of SEQ ID NO: 57 and the antisense strand of SEQ ID NO: 58, the double-stranded RNA consisting of the sense strand of SEQ ID NO: 59 and the antisense strand of SEQ ID NO: 60, the double-stranded RNA consisting of the sense strand of SEQ ID NO: 87 and the antisense strand of SEQ ID NO: 88, the double-stranded RNA consisting of the sense strand of SEQ ID NO: 89 and the antisense strand of SEQ ID NO: 90, the double-stranded RNA consisting of the sense strand of SEQ ID NO: 91 and the antisense strand of SEQ ID NO: 92, the double-stranded RNA consisting of the sense strand of SEQ ID NO: 93 and the antisense strand of SEQ ID NO: 94, the double-stranded RNA consisting of the sense strand of SEQ ID NO: 111 and the antisense strand of SEQ ID NO: 112, the double-stranded RNA consisting of the sense strand of SEQ ID NO: 113 and the antisense strand of SEQ ID NO: 114, the double-stranded RNA consisting of the sense strand of SEQ ID NO: 123 and the antisense strand of SEQ ID NO: 124, the double-stranded RNA consisting of the sense strand of SEQ ID NO: 125 and the antisense strand of SEQ ID NO: 126, the double-stranded RNA consisting of the sense strand of SEQ ID NO: 127 and the antisense strand of SEQ ID NO: 128, and the double-stranded RNA consisting of the sense strand of SEQ ID NO: 129 and the antisense strand of SEQ ID NO: 130.
